(19) United States
(12) Patent Application Publication
(10) Pub. No.: US 2024/0268892 A1
(43) Pub. Date: Aug. 15, 2024

(54) Virtual Reality Surgical Systems And Methods Including Virtual Navigation

(71) Applicant: MAKO Surgical Corp., Weston, FL (US)

(72) Inventor: Jared Leichner, North Miami Beach, FL (US)

(73) Assignee: MAKO Surgical Corp., Weston, FL (US)

(21) Appl. No.: 18/440,100

(22) Filed: Feb. 13, 2024

Related U.S. Application Data
(60) Provisional application No. 63/445,477, filed on Feb. 14, 2023.

(51) Int. Cl. A61B 34/10; G06F 3/01; G06T 15/10; G06T 19/00; G09B 23/28; G16H 20/40

(52) U.S. Cl. CPC A61B 34/10; G06F 3/016; G06T 15/10; G06T 19/003; G09B 9/00; G09B 23/28; G16H 20/40; A61B 2034/104; A61B 2034/105; A61B 2034/107; G06T 2210/41

(57) ABSTRACT

Virtual reality surgical systems, methods, and software for simulating surgical navigation. A head-mounted device includes a display positionable in front of eyes of a user. The display of the head-mounted device presents a virtual environment including therein a virtual surgical object, a virtual display device, and a virtual navigation system including a virtual localizer unit. A spatial relationship between the virtual localizer unit and the virtual surgical object is determined and a virtual representation of the virtual surgical object is presented on the virtual display device. A pose of the virtual representation is based on the determined spatial relationship between the virtual localizer unit and the virtual surgical object.

### CROSS-REFERENCE TO RELATED APPLICATIONS

The subject application claims priority to and all the benefits of U.S. Provisional Patent App. No. 63/445,477, filed Feb. 14, 2023, the contents of which are hereby incorporated by reference in their entirety.

### BACKGROUND

Virtual reality surgical simulators have been developed to provide training or simulation of surgical operations. A user wears a virtual reality headset and virtually interacts with a virtual operating room environment to provide a simulated experience such as operating virtual surgical tools.

Surgical navigation has become a significant aspect of modern surgery. Surgical navigation is the (real world) process by which surgical objects, such as tools or the patient, are tracked in the operating room using a tracking system. The tracking system can utilize the position between these surgical objects to facilitate surgical functions. For example, the tracking system can enable registration of a surgical plan to the physical anatomy of the patient and display a representation of the tool relative to the anatomy to provide a surgeon with guidance relative to the surgical plan. The display is typically a physical monitor or a screen in the operating room.

Although prior virtual reality surgical simulators may provide the user with a simulated experience in the operating room, such prior systems do not simulate surgical navigation. For example, some prior systems may virtually display components of a navigation system in the virtual operating room, such as a virtual camera or a virtual display. However, these prior systems virtually display such navigation system components merely for aesthetic or ornamental reasons, i.e., to provide an appearance of the operating room setting. These virtual navigation system components have no function whatsoever in the virtual reality simulation, and therefore, provide no training or simulation value for the user. Accordingly, with respect to navigation, prior virtual reality surgical simulators fall short of providing an accurate or complete experience for the user.

### SUMMARY

This Summary introduces a selection of concepts in a simplified form that are further described below in the Detailed Description below. This Summary is not intended to limit the scope of the claimed subject matter nor identify key features or essential features of the claimed subject matter.

In a first aspect, a virtual reality surgical system is provided. The virtual reality surgical system comprises a display positionable in front of eyes of a user; and at least one processor configured to: provide, on the display, a virtual environment and a virtual surgical object, and a virtual navigation system located within the virtual environment, wherein the virtual navigation system is configured to virtually track the virtual surgical object within the virtual environment.

In a second aspect, a virtual reality surgical system is provided. The virtual reality surgical system comprises a head-mounted device comprising a display positionable in front of eyes of a user; and at least one processor configured to: provide, on the display of the head-mounted device, a virtual environment and a virtual surgical object, a virtual display device, and a virtual navigation system located within the virtual environment, the virtual navigation system including a virtual localizer unit; determine a spatial relationship between the virtual localizer unit and the virtual surgical object in the virtual environment; and display a virtual representation of the virtual surgical object on the virtual display device, wherein a pose of the virtual representation is based on the determined spatial relationship between the virtual localizer unit and the virtual surgical object in the virtual environment.

In a third aspect, a virtual reality surgical system is provided. The virtual reality surgical system comprises a display positionable in front of eyes of a user; and at least one processor configured to: provide, on the display, a virtual environment and a virtual surgical object, a virtual display device, and a virtual navigation system located within the virtual environment; and evaluate a trackability of the virtual surgical object relative to the virtual navigation system.

In a fourth aspect, a virtual reality surgical system is provided. The virtual reality surgical system comprises a display positionable in front of eyes of a user; and at least one processor configured to: provide, on the display, a virtual environment and a virtual cutting tool and a virtual bone located within the virtual environment; and simulate virtual removal of portions of the virtual bone with the virtual cutting tool based on input from the user to control the virtual cutting tool.

In a fifth aspect, a virtual reality surgical system is provided. The virtual reality surgical system comprises a display positionable in front of eyes of a user; and at least one processor configured to: provide, on the display, a virtual environment and a virtual registration tool, a virtual bone, and a virtual navigation system located within the virtual environment; and simulate virtual registration of virtual bone to the virtual navigation system based on input from the user to control the virtual registration tool.

In a sixth aspect, a virtual reality surgical system is provided. The virtual reality surgical system comprises a display positionable in front of eyes of a user; and at least one processor configured to: provide, on the display, a virtual environment and a virtual surgical object and a virtual localizer located within the virtual environment; and enable the user to modify the pose of one or both of the virtual surgical object and the virtual localizer to simulate setting up a working relationship between the virtual surgical object and the virtual localizer in the virtual environment.

In a seventh aspect, a virtual reality surgical system is provided. The virtual reality surgical system comprises a display positionable in front of eyes of a user; and at least one processor configured to: provide, on the display, a virtual environment including within the virtual environment: a virtual manipulator with a virtual manipulator base, a virtual robotic arm coupled to the virtual manipulator base, and a virtual tool attached to the virtual robotic arm, and a virtual navigation system including a virtual base tracker attached to the virtual manipulator base and a virtual tool tracker attached to the virtual tool; and enable the user to move the virtual tool and virtual tool tracker pursuant to a registration process to simulate establishment of a virtual relationship between the virtual base tracker relative to the virtual manipulator base.

In an eighth aspect, a virtual reality surgical system is provided. The virtual reality surgical system comprises a display positionable in front of eyes of a user; and at least one processor configured to: provide, on the display, a virtual environment and a virtual tool and a virtual boundary located within the virtual environment; and simulate, based on input from the user to move the virtual tool, constraint of the virtual tool in response to the virtual tool interacting with the virtual boundary.

In a ninth aspect, a virtual reality surgical system is provided. The virtual reality surgical system comprises a display positionable in front of eyes of a user; and at least one processor configured to: provide, on the display, a virtual environment and a virtual surgical object located within the virtual environment, wherein the virtual surgical object is disassembled; and enable the user to virtually simulate assembly of the virtual surgical object.

A method of operating the virtual reality surgical system of any aspect is provided. A non-transitory computer readable medium being configured to implement the virtual reality surgical system of any aspect is provided.

Any of the above aspects can be combined in part or in whole.

For any of the above aspects, any one or more of the following implementations are contemplated, individually or in combination:

In one implementation, the at least one processor is configured to: provide the virtual navigation system to further include a virtual surgical object tracker coupled to the virtual surgical object within the virtual environment; and determine the spatial relationship between the virtual localizer unit and the virtual surgical object in the virtual environment by being configured to combine a first spatial relationship determined between the virtual localizer unit and the virtual surgical object tracker with a known spatial relationship between the virtual surgical object tracker and the virtual surgical object.

In one implementation, the at least one processor is configured to: further provide, on the display of the head-mounted device, a virtual patient anatomy located within the virtual environment; determine a spatial relationship between the virtual localizer unit and the virtual patient anatomy in the virtual environment; and display a virtual representation of the virtual patient anatomy on the virtual display device. In one implementation, the at least one processor is configured to receive image data of actual patient anatomy and to provide the virtual patient anatomy based on the image data of actual patient anatomy.

In one implementation, the at least one processor is configured to: provide the virtual navigation system to further including a virtual anatomy tracker coupled to the virtual patient anatomy within the virtual environment; and determine the spatial relationship between the virtual localizer unit and the virtual patient anatomy in the virtual environment by being configured to combine a first spatial relationship determined between the virtual localizer unit and the virtual anatomy tracker with a known spatial relationship between the virtual anatomy tracker and the virtual patient anatomy.

In one implementation, the at least one processor is configured to: define a coordinate system of the virtual environment; determine coordinates of the virtual localizer unit and coordinates of the virtual surgical object relative to the coordinate system of the virtual environment; and determine the spatial relationship between the virtual localizer unit and the virtual surgical object by being configured to compare the coordinates of the virtual localizer unit and coordinates of the virtual surgical object relative to the coordinate system of the virtual environment.

In one implementation, the at least one processor is configured to: provide the virtual localizer unit with a virtual field of view; determine coordinates of the virtual localizer unit and coordinates of the virtual surgical object relative to the virtual field of view; and determine the spatial relationship between the virtual localizer unit and the virtual surgical object by being configured to compare the coordinates of the virtual localizer unit and coordinates of the virtual surgical object relative to the virtual field of view. In one implementation, the at least one processor is configured to: display the virtual representation of the virtual surgical object on the virtual display device in response to the virtual surgical object entering the virtual field of view of the virtual localizer unit; and prevent the display of the virtual representation of the virtual surgical object on the virtual display device in response to the virtual surgical object exiting the virtual field of view of the virtual localizer unit.

In one implementation, the virtual surgical object is further defined as a virtual probe, and wherein the at least one processor is configured to: receive an input from the user to control a position of the virtual probe within the virtual environment; and register the virtual patient anatomy in response to the virtual probe collecting points on a surface of the virtual patient anatomy based on the input from the user. In one implementation, the at least one processor is configured to: display, on the virtual display device, the virtual representation of the virtual patient anatomy, and points to be collected on the surface of the virtual representation of the virtual patient anatomy; display, on the virtual display device, the virtual representation of the virtual surgical object relative to the virtual representation of the virtual patient anatomy during collection of points on the surface; and display, on the virtual display device, a notification or alert indicative of completion of a proper registration of the virtual patient anatomy. In one implementation, the virtual reality surgical system further comprises a haptic device configured to provide haptic feedback to the user in response to the virtual probe collecting points on the surface of the virtual patient anatomy.

In one implementation, the virtual surgical object is further defined as a virtual cutting tool, and wherein the at least one processor is configured to: receive an input from the user to control a position and an operation of the virtual cutting tool within the virtual environment; and enable the virtual cutting tool to perform a virtual cutting of the virtual patient anatomy based on user control of the virtual cutting tool. In one implementation, the at least one processor is configured to: display, on the virtual display device, the virtual representation of the virtual cutting tool relative to the virtual representation of the virtual patient anatomy during the virtual cutting of the virtual patient anatomy; and display, on the virtual display device, feedback related to the virtual cutting of the virtual patient anatomy. In one implementation, the virtual cutting tool is further defined as a virtual hand-held cutting tool or a virtual robotic manipulator. In one implementation, the virtual reality surgical system further comprises a haptic device configured to provide haptic feedback to the user, and wherein the at least one processor is configured to: define a virtual boundary relative to the virtual patient anatomy, the virtual boundary delineating a region of the virtual patient anatomy to be cut by the virtual cutting tool from another region of the virtual patient anatomy to be avoided by the virtual cutting tool; and detect that the virtual cutting tool has met or exceeded the virtual boundary; and in response, cause the haptic device to provide haptic feedback to the user.

In one implementation, the display of the head-mounted device is configured to display instructions for assembling the virtual surgical object.

In one implementation, the display is of a head-mounted device. In one implementation, the display surrounds the head of the user but is not mounted to the head of the user. In another implementation, the display is of a table or floor console system that the user approaches.

In one implementation, the virtual reality surgical system further comprises a camera having a field of view to capture image data of a hand of the user, and wherein the at least one processor is configured to position the virtual surgical object in the virtual environment based on the image data of the hand of the user. In one implementation, the virtual reality surgical system further comprises a user input configured to detect a motion of a hand of the user, and wherein the at least one processor is configured to position the virtual surgical object in the virtual environment based on the detected motion of the hand of the user.

In one implementation, the at least one processor is configured to: provide the virtual navigation system to further include a virtual tracker within the virtual environment;

determine the spatial relationship between the virtual localizer unit and the virtual tracker in the virtual environment; and display, on the virtual display device, feedback related to the spatial relationship between the virtual localizer unit and the virtual tracker in the virtual environment.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of a virtual reality surgical system including a head-mounted device including a display, wherein a virtual environment displayed on the display is shown, according to one example.

FIG. 2 is a schematic view of the virtual reality surgical system, according to one example

FIG. 3 illustrates steps of tracking a hand of a user of the virtual reality surgical system and positioning a virtual surgical object within the virtual environment, according to one implementation.

FIG. 4 is a perspective view of an example virtual environment, wherein a spatial relationship between a virtual localizer unit and a virtual pointer is shown.

FIG. 5 is a perspective view of an example virtual environment, wherein a spatial relationship between a virtual localizer unit and a virtual handheld surgical tool is shown.

FIGS. 6 and 7 are example views of the virtual environment as displayed through the display of the head-mounted device, wherein a user registers a virtual patient anatomy with a virtual pointer.

FIGS. 8A and 8B are schematic views of a virtual localizer unit and a virtual surgical object, wherein a field of view of the virtual localizer unit is shown, according to certain implementations.

FIGS. 9A and 9B are schematic views of a virtual localizer unit and a virtual surgical object, wherein a field of view of the virtual localizer unit and a field of view of the virtual surgical object are shown, according to certain implementations.

FIG. 10 is a view of an example virtual environment as displayed through the display of the head-mounted device, wherein a virtual pointer is prevented from being displayed on a virtual display, e.g., due the virtual pointer being beyond a field of view of the virtual localizer.

FIG. 11 is a view of an example virtual environment as displayed through the display of the head-mounted device, wherein a user performs cutting of a virtual patient anatomy with a virtual handheld surgical tool, according to one implementation.

FIG. 12 is a schematic view of a virtual localizer unit, wherein a field of view and a tracking quality of the virtual localizer unit are shown.

### DETAILED DESCRIPTION

### I. Overview of the Virtual Reality Surgical System

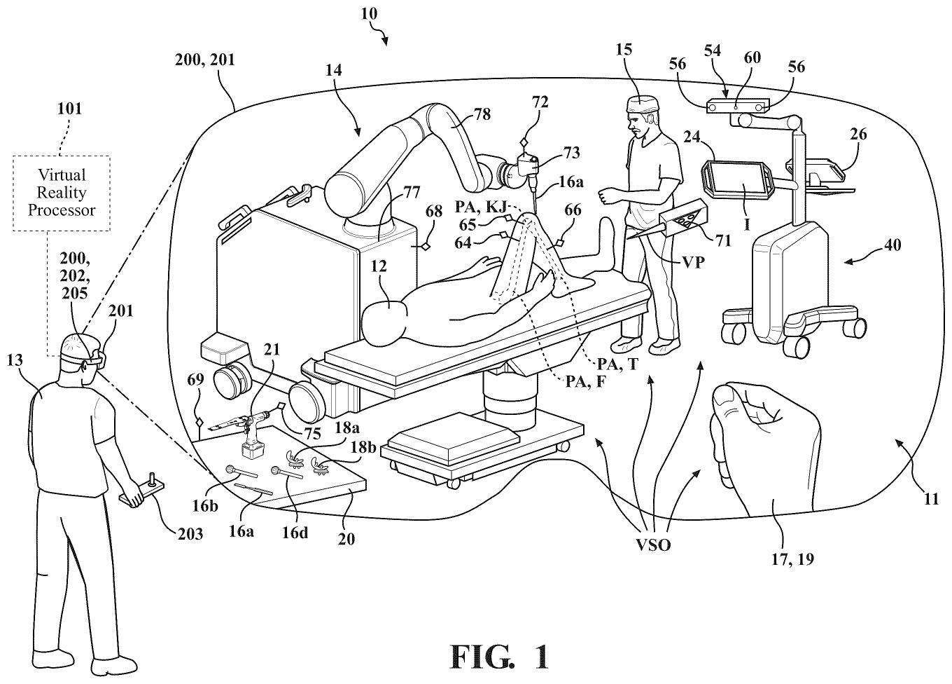

FIG. 1 depicts a virtual reality surgical system 10, which may be used for enabling one or more members of a surgical team to virtually experience aspects of surgery or surgical workflow. The virtual reality surgical system 10 can be used for training purposes, planning purposes, surgical procedure simulation purposes, inventory management, component assembly, component inspection, or any combination thereof.

The virtual reality surgical system 10 includes a display 201 positionable in front of eyes of a user 13 of the virtual reality surgical system 10. In one example, the display 201 of a head-mounted device (HMD) 200 and the display 201 is presented in front of the eyes of the user 13 when the user 13 wears the HMD 200. The display 201 may comprise a liquid crystal display, liquid crystal on silicon display, organic light-emitting diode display, or any equivalent type of display to provide the user with an immersive experience. The HMD 200 may include a head-mountable structure 202, which may be in the form of an eyeglass and may include additional headbands or supports to hold the HMD 200 on a head of the user. In other instances, the HMD 200 may be integrated into a helmet or other structure worn on the user's head, neck, and/or shoulders. In another implementation, instead of being on the HMD 200, the display 201 may surround the head of the user 13 without being mounted to the head of the user. For example, the display 201 may be a dome that is lowered over the user's head to provide a 180 to 360 degree view. Alternatively, the display 201 may be of a table or a floor-mounted console system with an interface into which the user looks.

The virtual reality surgical system 10 also includes a virtual reality processor 101, which may display a virtual environment 11 on the display 201. In the instance of FIG. 1, the virtual environment 11 is represented as a virtual surgical operating room. The virtual reality processor 101 displays the virtual environment 11 from a first-person perspective of the user 13, as a means of immersing the user 13 in the virtual environment 11.

A virtual reality processor 101 may communicate with the HMD 200 as well as other components, such as a hand controller 203 held by the user 13 of the virtual reality surgical system 10, a microphone, and/or a speaker within a proximity of the user 13 to provide the user 13 with feedback corresponding to the virtual feedback provided in the virtual environment 11 and/or to receive inputs from the user 13 for interacting with the virtual environment 11.

### II. Overview of the Virtual Surgical Environment

As previously stated, the virtual reality processor 101 provides the virtual environment 11 on the display 201 of the HMD 200. As shown in FIG. 1, the virtual environment 11 may be a virtual surgical operating room. Additionally, the virtual reality processor 101 may provide a variety of virtual surgical objects VSO within the virtual environment 11, which could be any virtual object located in a virtual surgical operating room.

For example, the virtual surgical objects VSO may include a virtual patient 12 having a virtual patient anatomy PA within the virtual environment 11. The virtual surgical objects VSO may also include a virtual localizer unit 54, a virtual manipulator 14, a virtual pointer VP, a virtual handheld surgical tool 21, virtual surgical tools 16a, 16b, 16c, 16d of various types, shapes, and/or sizes, virtual implants 18a, 18b of various types, shapes, and/or sizes, a virtual object surgical tracker 64, 65, 66, 68, 72, 75, a virtual circulating table 20, a virtual participant 15, the virtual representation 17 of the user, and the like. Any of the virtual objects described herein within the virtual environment 11 may be identified as a virtual surgical object VSO.

An aesthetic backdrop or layout of the virtual surgical operating room may be selected or loaded by the virtual reality processor 101 from a library or a database. The database may comprise a plurality of virtual surgical operating rooms that mimic real world operating rooms to enhance the user's experience. The virtual surgical operating room may correspond to the actual surgical operating room that the user is expected to utilize. Any of the virtual surgical objects VSO described above may also be loaded with the corresponding virtual surgical operating room.

The virtual reality processor 101 may be configured to provide a virtual patient anatomy PA of the virtual patient 12. The virtual reality processor 101 may be configured to provide any suitable virtual patient anatomy PA. For example, referring to FIG. 1, the virtual reality processor 101 provides a virtual bone as the virtual patient anatomy PA, such as a virtual femur F or a virtual tibia T of the virtual patient 12. Other types of soft or hard tissue anatomy may be virtually represented, such as a virtual knee joint KJ (shown in FIG. 1), a virtual hip joint, a virtual shoulder joint, a virtual vertebra or spine, a virtual tumor, virtual soft tissue structures, virtual ligaments, virtual veins or arteries, or the like. In some instances, the virtual reality processor 101 may be configured to receive image data of actual patient anatomy, such as image data of an actual femur and/or an actual tibia of an actual patient. The virtual reality processor 101 may then generate the virtual patient anatomy PA based on the image data of the actual patient anatomy. The virtual reality processor 101 may be configured to receive or generate a 3D model of the actual anatomy based on the preoperative images. This 3D model may be utilized to generate the virtual reality model. In this way, the virtual patient anatomy PA generated by the virtual reality processor 101 may be realistic and may also be patient-specific, which may be beneficial in instances where the user 13 uses the virtual reality surgical system 10 as a means of preparing for performing surgery on a specific patient. In other examples, the virtual reality processor 101 may utilize generic anatomies or patient geometries to generate the virtual patient 12 and virtual patient anatomy PA. In one example, the virtual reality processor 101 may have access to a database of a virtual patient and virtual anatomy sizes. Information about the patient, such as weight, height, BMI, etc., may be inputted into the virtual reality processor 101. Based on this patient information, the virtual reality processor 101 can choose the appropriately sized virtual patient 12 and a size of the virtual patient anatomy PA for the virtual simulation.

The virtual reality processor 101 may provide various virtual surgical object trackers and virtual anatomy trackers coupled to the virtual surgical objects VSO and virtual patient anatomies PA within the virtual environment 11. The various virtual surgical object trackers may be located and tracked by the virtual reality processor 101 to determine virtual locations (e.g., virtual positions and/or virtual orientations) of the virtual surgical objects VSO. In the instance illustrated in FIG. 1, one virtual anatomy tracker 64 is affixed to the virtual femur F of the virtual patient 12, one virtual anatomy tracker 65 is affixed to the virtual knee joint KJ of the virtual patient 12, and another virtual anatomy tracker 66 is affixed to the virtual tibia T of the virtual patient 12. Additionally, a virtual surgical object trackers 68, 72 are shown coupled to a virtual base and to a virtual tool 73, respectively, of the virtual manipulator 14. A virtual surgical object tracker 71 is coupled to the virtual pointer VP, a virtual surgical object tracker 69 is coupled to the virtual circulating table 20, and a virtual surgical object tracker 75 is coupled to the virtual handheld surgical tool 21. It should be understood that, in other instances, the virtual environment 11 may include additional virtual surgical object tracker for tracking additional virtual surgical objects VSO. For instance, the virtual environment 11 may include the virtual surgical object tracker 71 coupled to one or more of the virtual surgical tools 16a, 16b, 16c, 16d and one or more of the virtual implants 18a, 18b. These virtual trackers can be virtual optical (active or passive) trackers, virtual magnetic trackers, virtual radio frequency trackers, virtual ultrasound trackers, virtual inertial trackers, or any combination thereof.

The virtual reality processor 101 may provide a virtual surgical navigation system 40 within the virtual environment 11. The trackers are part of the virtual surgical navigation system 40. The virtual navigation system 40 may serve as a reference point for the virtual reality processor 101 when the virtual reality processor 101 determines the virtual locations (e.g., virtual positions and/or virtual orientations) of the virtual surgical objects VSO. In the instance of FIG. 1, the virtual navigation system 40 includes a virtual localizer unit 54, which is represented as a virtual optical localizer 54 including virtual camera units 56. The virtual localizer unit 54 is provided on a virtual navigation cart in FIG. 1. The virtual localizer unit 54 may be a virtual electromagnetic localizer, a virtual radiofrequency localizer, a virtual machine vision localizer, a virtual ultrasound localizer, or any other suitable virtual localizer. The virtual reality processor 101 may determine a spatial relationship between the virtual localizer unit 54 and the virtual surgical objects VSO when determining the virtual locations of the virtual surgical objects VSO.

The virtual reality processor 101 may provide a plurality of virtual display devices 24, 26 within the virtual environment 11. The virtual display devices 24, 26 may be strategically placed within the virtual environment 11 such that the virtual display devices 24, 26 may be viewed by the user 13 of the virtual reality surgical system 10. The virtual display devices 24, 26 may be represented as any suitable form of display, including one or more displays attached to a virtual navigation system 40 cart, or displays of portable electronic devices (e.g., tablets, smart phones, etc.) that are virtually held by the user 13 in the virtual environment 11. The virtual reality processor 101 may display a virtual representation of a virtual surgical object VSO on the virtual display devices 24, 26 based on the determined spatial relationship between the virtual localizer unit 54 and the virtual surgical object VSO.

The virtual reality processor 101 may provide a virtual representation 17 of the user 13. For example, as shown in FIG. 1, the virtual reality processor 101 provides a virtual representation 17 of the user 13, of which a virtual hand 19 corresponding to a hand of the user 13 is shown. Depending on a perspective of the user 13, a greater or lesser number of features of the virtual representation 17 may be shown. For example, in instances where the user 13 is looking down, a virtual leg of the virtual representation 17 may be shown. In instances where the user 13 is looking up, no feature of the virtual representation 17 may be shown.

In some instances, the virtual displays 24, 26 may be represented as virtual touchscreen displays and may serve as virtual input devices (I) configured to receive an input from the user 13 of the virtual reality surgical system 10. In this way, the user 13 of the virtual reality surgical system 10 may interact with the virtual input devices (I) to input information into the virtual reality surgical system 10. In other instances, the virtual input devices (I) may be represented as a virtual keyboard and/or a virtual mouse. Other virtual input devices (I) are contemplated including a virtual touch screen, as well as voice and/or gesture activation, and the like. The virtual reality processor 101 may also provide other virtual visual feedback devices such as virtual laser pointers, virtual laser line/plane generators, virtual LEDs, and other virtual light sources within the virtual environment.

In some instances, the virtual reality processor 101 may provide virtual participants 15 of the surgical team within the virtual environment 11. For example, the virtual reality processor 101 may provide a virtual surgeon, a virtual assistant, a virtual circulating nurse, a virtual scrub nurse, and a virtual operating room (OR) technician ORT. Other types/numbers of virtual participants are also contemplated. The virtual participants 15 may be controlled by the virtual reality processor 101 to virtually perform tasks within a surgical workflow. In instances where more than one user 13 simultaneously uses the virtual reality surgical system 10, the virtual reality processor 101 may provide virtual participants 15 as virtual representations of the more than one user 13 of the virtual reality surgical system 10. In such instances, the virtual reality processor 101 may control the virtual participant 15 based on inputs received from the more than one user 13 of the virtual reality surgical system 10.

It is contemplated that more than one user may simultaneously use the virtual reality surgical system 10. For example, in an instance where a first user and a second user simultaneously use the virtual reality surgical system 10, the virtual reality surgical system 10 may include a first HMD 200 including a first display 201 positionable in front of eyes of the first user, as well as a second HMD 200 including a second display 201 positionable in front of eyes of the second user. The virtual reality processor 101 may display the virtual environment 11 on each display 201 from a vantage point of the corresponding user. Additionally, the virtual reality processor 101 may provide virtual representations 15 of each user within the virtual environment 11. As such, first and second users of the virtual reality surgical system 10 may each interact with the virtual environment 11 and with one another within the virtual environment 11.

### III. Overview of the Virtual Reality Processor

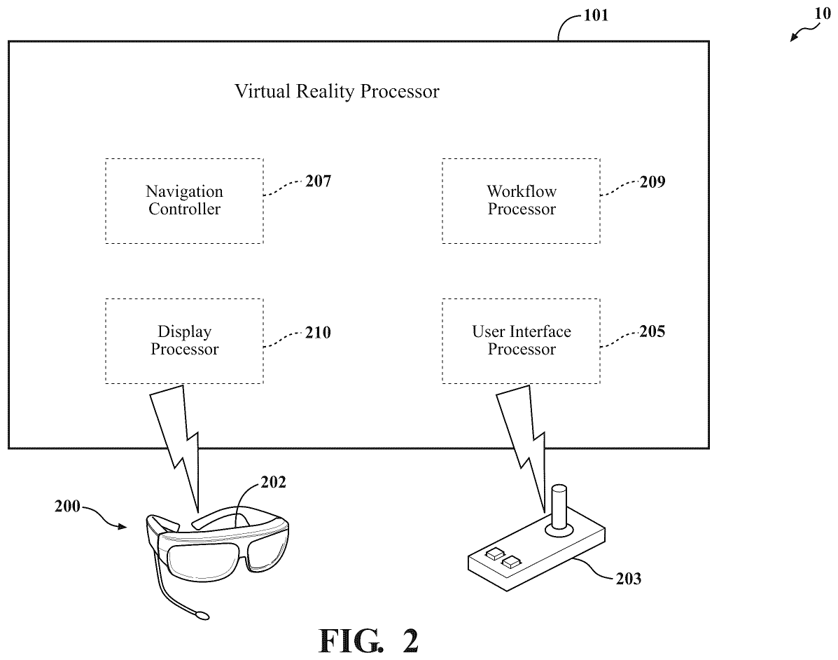

As previously described, the virtual reality surgical system 10 includes the virtual reality processor 101, shown diagrammatically in FIG. 2. The virtual reality processor 101 is shown as being divided into several sub-processors or controllers 205, 207, 209, 210 for facilitating the various operations of the virtual reality processor 101. As each of the sub-processors are a part of the virtual reality processor 101, any of the sub-processors are able to communicate with one another.

The virtual reality surgical system 10 is shown in FIG. 2 as including a single virtual reality processor 101. However, in other instances, the virtual reality surgical system 10 may include more than one virtual reality processor 101. In such instances, the more than one virtual reality processor 101 may be configured to perform the operations described herein individually or collectively. The virtual reality processor 101 and described controllers can access software instructions stored on a non-transitory computer readable medium or memory and execute the software instructions for performing the various functionality described herein.

As shown in FIG. 2, the virtual reality processor 101 may include a display processor 210 in communication with the HMD 200. The display processor 210 may be configured to generate the virtual environment 11 and provide the virtual environment 11 on a display, such as the display 201 of the HMD 200. For example, the display processor 210 may be configured to generate the above-described virtual environment 11, as well as the virtual surgical objects VSO within the virtual environment 11, such as a virtual patient anatomy PA, the virtual localizer unit 54, the virtual manipulator 14, the virtual handheld surgical tool 21, the virtual pointer VP, the virtual surgical tools 16a, 16b, 16c, 16d, the virtual implants 18a, 18b, the virtual object surgical tracker 64, 66, 68, 72, 75, the virtual circulating table 20, the virtual participant 15, the virtual representation 17 of the user, and the like.

The display processor 210 may be configured to generate the virtual environment 11 and provide the virtual environment 11 on a display using any suitable frame rate. For example, the display processor 210 may be configured to generate the virtual environment 11 based on a rate of 60 frames per second, 72 frames per second, 90 frames per second, 120 frames per second, 144 frames per second, or any other suitable frame. In instances where the display processor 210 provides the virtual environment 11 on the display 201 of the HMD 200, the display processor 210 may be configured to generate the virtual environment 11 based on a rate that realistically displays changes in the virtual environment 11. For example, the display processor 210 may generate and display the virtual environment 11 at a frame rate that enables motion of the various objects in the virtual environment 11 to appear seamless to the user 13.

In instances where the display processor 210 provides the virtual environment 11 on the display 201 of the HMD 200, the display processor 210 may be configured to provide the virtual environment 11 on the display 201 based on tracking a location of the HMD 200. The pose of the HMD 200 may be defined based on an HMD coordinate system (HCS). The HMD coordinate system may be defined in the real-world based on internal and/or external sensors related to the HMD 200. For example, an external (real) tracking system separate from the HMD 200 may track the pose of the HMD 200. Additionally, or alternatively, the HMD may comprise inertial sensors (IMUs) to detect the pose of the HMD 200 relative to the HMD coordinate system. The tracking sensors of the HMD 200 may comprise IR depth sensors, to layout the space surrounding the HMD 200, such as using structure-from-motion techniques or the like. A camera may also be mounted to the HMD 200 to detect the external (real world) environment surrounding the HMD 200. Based on any of these inputs, if the user 13 changes the pose of the HMD 200 within the HMD coordinate system, the display processor 210 updates the display of the virtual environment 11 to correspond to the motions of the pose of the HMD 200.

The display processor 210 may also be configured to receive an input signal from the HMD 200 corresponding to an input from the user 13. In this way, the user 13 may control the HMD 200, the display processor 210, and/or other sub-processors of the virtual reality processor 101. For example, the HMD 200 may include a user interface, such as a touchscreen, a push button, and/or a slider. In one instance, the user 13 may press the push button to cease actuation of the virtual handheld surgical tool 21. In such an instance, the HMD 200 receives the input and transmits a corresponding input signal to the display processor 210. The display processor 210 then generates the virtual environment 11, wherein actuation of the virtual handheld surgical tool 21 is ceased. As another example, the HMD 200 may include an infrared motion sensor to recognize gesture commands from the user 13. The infrared motion sensor may be arranged to project infrared light or other light in front of the HMD 200 so that the motion sensor is able to sense the user's hands, fingers, or other objects for purposes of determining the user's gesture command. In another example, the HMD 200 may be configured to capture image data of a hand of the user 13 to determine a position of the hand of the user 13. In yet another example, the HMD 200 may include a microphone to receive voice commands from the user 13. In one instance, when the user 13 speaks into the microphone, the microphone receives the voice command and transmits a corresponding input signal to the display processor 210.

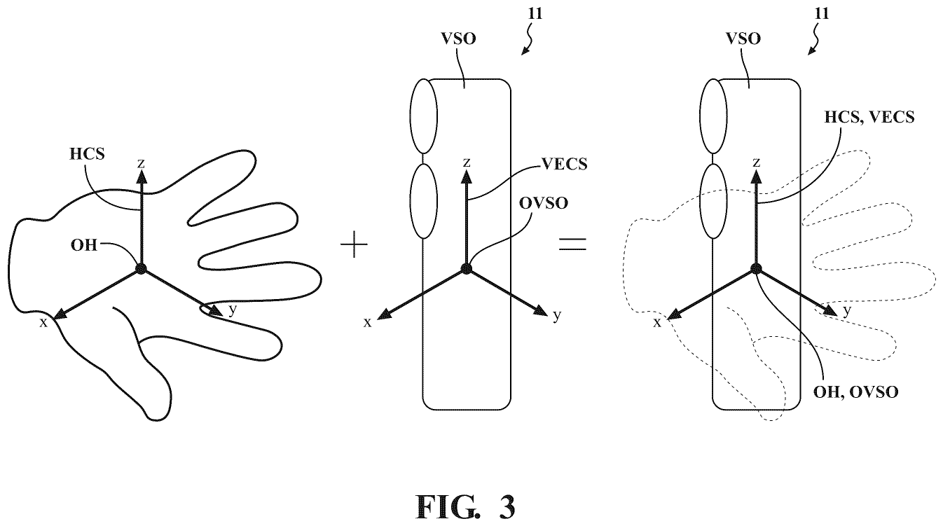

For example, referring to FIG. 3, the display processor 210 may be configured to receive an input signal from the HMD 200 as the HMD 200 tracks a hand of a user 13. Once the display processor 210 receives the input signal, the display processor 210 may generate and display a virtual surgical object VSO within the virtual environment 11 (e.g., the virtual handheld surgical tool 21) in a manner that mimics a motion of the hand of the user 13. Specifically, as shown in FIG. 3, the HMD 200 may track an origin OH of the hand of the user 13 in the coordinate system (HCS) of the HMD 200 such that, as the user 13 moves their hand, the HMD 200 tracks the origin OH of the hand and generates a corresponding input signal. Additionally, the display processor 210 may assign the origin OH of the hand to an origin OVSO of the virtual surgical object VSO in a virtual coordinate system VECS of the virtual environment 11. In this way, the display processor 210 may generate and display the virtual surgical object VSO based on the input signal such that motion of the origin OVSO of the virtual surgical object VSO in the virtual coordinate system VECS mimics the motion of the origin OH of the hand in the coordinate system HCS. Additionally, the display processor 210 may generate and display the virtual hand 19 of the user 13 such that the origin of the virtual hand 19 in the virtual coordinate system VECS corresponds to the origin OH of the hand of the user 13 in the coordinate system HCS of the HMD 200. In some instances, the hand is virtually provided in the surgical simulation and the virtual hand moves according to the input signal from the HMD 200. Once the virtual hand reaches the virtual surgical object VSO in the virtual environment 11, the assignment can be made enabling the user to control the virtual surgical object VSO. The assignment can be made by the display processor 210 detecting the user closing their hand, i.e., thereby mimicking the grasping of the virtual surgical object VSO. Alternatively, once the virtual hand is over the virtual surgical object VSO, the display processor 210 can receive an input from the user to grasp the virtual surgical object VSO.

The display processor 210 may also be configured to provide feedback to the user 13 via the HMD 200. The HMD 200 may include any suitable haptic (vibratory) and/or auditory feedback devices. In one instance, the display processor 210 may be configured to provide auditory feedback to the user 13 by transmitting a feedback signal to the HMD 200. For example, the display processor 210 may provide auditory feedback to the user 13 based on a virtual alert generated within the virtual environment 11. The user input device 203 receives the feedback signal and an auditory feedback device of the user input device 203, such as a speaker of within a proximity of the user 13, provides an audible alert to the user 13. Visual feedback can also be provided by alerts or notifications provided on the display 201.

As shown in FIG. 2, the virtual reality processor 101 may include a user input device processor 205. The user input device processor 205 may be configured to receive an input signal from a user input device, such as the hand controller 203 shown in FIG. 1.

The user input device 203 may include any suitable user interface, such as a touchscreen, a push button, and/or a joystick. The user 13 may interact with user interfaces of the user input device 203 to interact with the virtual environment 11. For example, in an instance where the hand controller 203 includes a push button, the user 13 may push the push button to pick up the virtual handheld surgical tool 21 in the virtual environment 11. When the user 13 pushes the push button, the user input device 203 receives the input and transmits a corresponding input signal to the user input device processor 205. The display processor 210 may then generate the virtual environment 11 such that the virtual handheld surgical tool 21 is picked up and provide the virtual environment 11 on the display 201 of the HMD 200.

In other instances, the user input device 203 may include a variety of sensors configured to transmit an input signal to the user input device processor 205. For example, the user input device 203 may include one or more inertial measurement units, such as 3-D accelerometers and/or 3-D gyroscopes, which may provide an input signal corresponding to a motion of the user input device 203 to the user input device processor 205. In such an instance, the user 13 may move the user input device 203 to mimic a desired motion of a virtual handheld surgical tool 21 in the virtual environment 11. The movement by the user 13 is detected by an inertial measurement unit, and the inertial measurement unit transmits an input signal to the user input device processor 205. The display processor 210 may then generate the virtual environment 11 such that the virtual handheld surgical tool 21 is moved in the desired manner and provide the virtual environment 11 on the display 201 of the HMD 200.

The user input processor 205 may also be configured to provide feedback to the user 13 via the user input device 203. The user input device 203 may include any suitable haptic and/or auditory devices. In one instance, the user input processor 205 may be configured to provide haptic feedback to the user 13 by transmitting a feedback signal to the user input device 203. A haptic feedback device of the user input device 203, such as a vibratory device, may then provide haptic feedback to the user 13.

As shown in FIG. 2, the virtual reality processor 101 may include a navigation processor 207. The navigation processor 207 may be configured to determine a spatial relationship between the virtual localizer unit 54 and a virtual surgical object VSO in the virtual environment 11. The navigation processor 207 can determine the spatial pose of the virtual patient anatomy PA, the virtual manipulator 14, the virtual handheld surgical tool 21, the virtual pointer VP, one or more of the virtual surgical tools 16a, 16b, 16c, 16d, one or more of the virtual implants 18a, 18b, a virtual object surgical tracker 64, 66, 68, 72, 75, the virtual circulating table 20, a virtual participant 15, the virtual representation 17 of the user, and the like. The navigation processor 207 may also be configured to determine a visibility of the virtual surgical object VSO, as well as a quality of virtual tracking the virtual surgical object VSO.

As shown in FIG. 2, the virtual reality processor 101 may include a workflow processor 209. The workflow processor 209 may be configured to assist one or more users 13 of the virtual reality surgical system 10 by generating and outputting information to the one or more users 13 regarding a pre-scripted surgical workflow. In some cases, the workflow processor 209 may generate visual, audible, and/or tactile aids based on a workflow step of a pre-scripted surgical workflow. The visual aids may be provided to the one or more users 13 virtually via the virtual environment 11 and/or via the HMD 200. For example, a visual aid may be provided to a user 13 via the virtual display 24 and/or via the display 201 of the HMD 200. The audible aids may be provided to the one or more users 13 via an auditory feedback device of the HMD 200, the hand controller 203, a speaker within a proximity of the user 13, and/or any other suitable auditory feedback device. The tactile aids may be provided to the one or more users 13 via a haptic feedback device of the HMD 200, the hand controller 203, and/or any other suitable haptic feedback device. The workflow processor 209 is configured to provide any of the functionality described in U.S. Pat. No. 11,114,199, entitled “Workflow Systems And Methods For Enhancing Collaboration Between Participants In A Surgical Procedure”, the contents of which are hereby incorporated by reference.

The virtual reality processor 101 may be a computer separate from the HMD 200, located remotely from the support structure 202 of the HMD 200, or may be integrated into the support structure 202 of the HMD 200. The virtual reality processor 101 may be a laptop computer, desktop computer, microcontroller, or the like with memory, one or more processors (e.g., multi-core processors), input devices, output devices (fixed display in addition to HMD 200), storage capability, etc. In other instances, the virtual reality processor 101 may be integrated into the user input device 203.

### IV. Operations of the Virtual Reality Surgical System

As described, the virtual reality surgical system 10 may be used to train a user 13 on aspects of surgery and/or to enable a user 13 to simulate a surgical procedure. For both of these purposes, the navigation processor 207 determines a spatial relationship between the virtual localizer unit 54 and the corresponding virtual surgical object VSO in the virtual environment 11.

### A. Determining Spatial Relationships

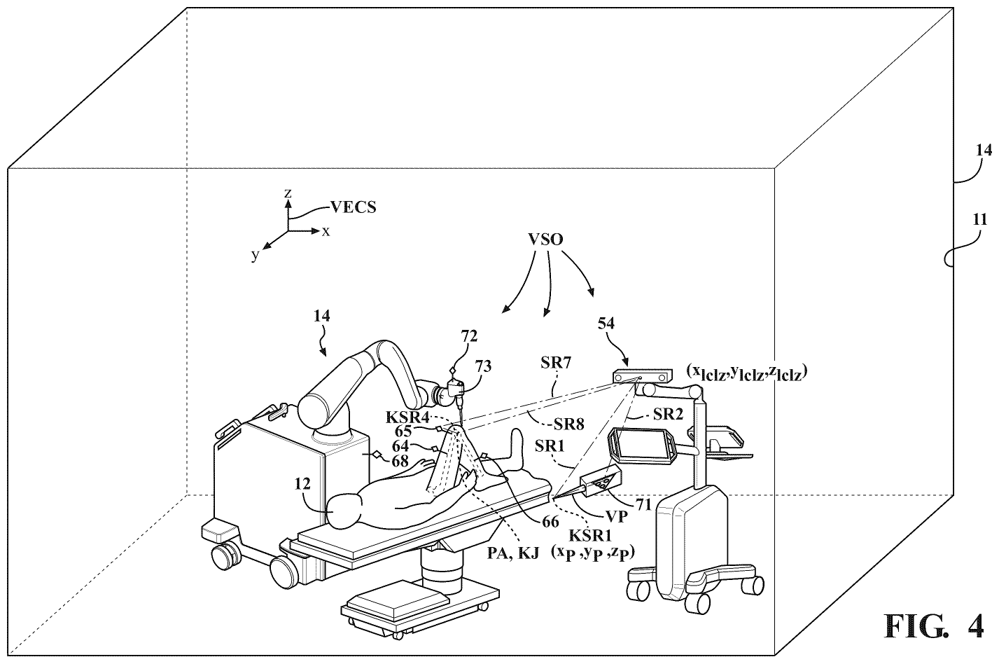

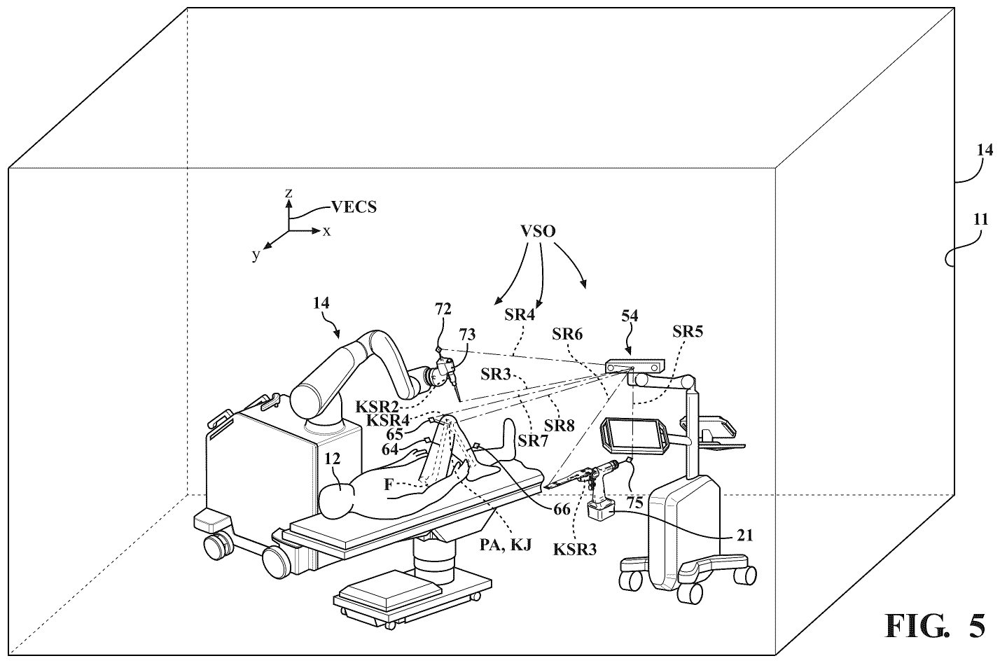

The navigation processor 207 can determine a spatial relationship between virtual surgical objects VSO. For example, the navigation processor 207 can determine various spatial relationships SR1-SR8 between virtual surgical objects VSO, the spatial relationships being shown in FIGS. 4 and 5. In FIG. 4, the virtual surgical objects VSO include the virtual localizer unit 54 and the virtual pointer VP and the navigation processor 207 can determine a spatial relationship SR1 between the virtual localizer unit 54 and the virtual pointer VP. In FIG. 5, the virtual surgical objects VSO include the virtual localizer unit 54 and the virtual surgical tool 73 of the virtual manipulator 14 and the navigation processor 207 can determines a spatial relationship SR3 between the virtual localizer unit 54 and the virtual surgical tool 73. In FIG. 5, the virtual surgical objects VSO include the virtual localizer unit 54 and the virtual handheld surgical tool 21 and the navigation processor 207 can determines a spatial relationship SR5 between the virtual localizer unit 54 and the virtual handheld surgical tool 21. In FIGS. 4 and 5, the virtual surgical objects VSO also include a virtual patient anatomy PA, illustrated as the virtual knee joint KJ of the virtual patient 12, and the navigation processor 207 can determine the spatial relationship SR7 between the virtual localizer unit 54 and the virtual knee joint KJ.

In one example, as shown in FIG. 4, the navigation processor 207 can determine the spatial relationship SR1 by determining a spatial relationship SR2 between the virtual localizer unit 54 and the virtual surgical object tracker 71 coupled to the virtual pointer VP and combining the determined spatial relationship SR2 with a known spatial relationship KSR1 between the virtual surgical object tracker 71 and the virtual pointer VP. In other examples, the spatial relationship SR1 between the virtual localizer unit 54 and the virtual pointer VP can be determined directly, without considering the tracker 71, i.e., without SR2, or KSR1. For example, the spatial relationship SR1 can be determined between the virtual localizer unit 54 and a virtual feature of the virtual pointer VP, such as the probe tip.

Referring to FIG. 5, the navigation processor 207 can determine the spatial relationship SR3 between the virtual localizer unit 54 and the virtual surgical tool 73 of the virtual manipulator 14. For instance, the navigation processor 207 may determine the spatial relationship SR3 between the virtual localizer unit 54 and a virtual feature of the virtual surgical tool 73, such as a tip of an end effector of the virtual surgical tool 73, as shown in FIG. 5. In one example, as shown, the navigation processor 207 determines the spatial relationship SR3 by determining a spatial relationship SR4 between the virtual localizer unit 54 and the virtual surgical object tracker 72 coupled to the virtual surgical tool 73 and combining the determined spatial relationship SR4 with a known spatial relationship KSR2 between the virtual surgical object tracker 71 and the virtual surgical tool 73. In other examples, the spatial relationship SR3 between the virtual localizer unit 54 and the virtual surgical tool 73 can be determined directly, without considering the tracker 72, i.e., without SR4, or KSR2. For example, the spatial relationship SR3 can be determined between the virtual localizer unit 54 and a virtual feature of the virtual surgical tool 73 or virtual manipulator 14. Additionally, virtual kinematic data related to the virtual manipulator 14 can be simulated to determine any relationship involving any components of the virtual manipulator 14, such as a virtual base of the virtual manipulator 14, a virtual link or joint of the virtual manipulator 14, and/or a virtual end effector of the virtual manipulator 14. The virtual kinematic data can be based on real world kinematic data information related to a physical manipulator. The virtual reality processor 101 may access the virtual kinematic data.

FIG. 5 also illustrates an instance where a virtual handheld surgical tool 21 is provided, wherein the navigation processor 207 can determine the spatial relationship SR5 between the virtual localizer unit 54 and the virtual handheld surgical tool 21. For instance, the navigation processor 207 may determine the spatial relationship SR5 between the virtual localizer unit 54 and a virtual feature of the virtual handheld surgical tool 21, such as a tip of a saw blade of the virtual handheld surgical tool 21, as shown in FIG. 5. As shown, the navigation processor 207 determines the spatial relationship SR5 by determining a spatial relationship SR6 between the virtual localizer unit 54 and the virtual surgical object tracker 75 coupled to the virtual handheld surgical tool 21 and combining the determined spatial relationship SR6 with a known spatial relationship KSR3 between the virtual surgical object tracker 75 and the virtual handheld surgical tool 21. Again, the spatial relationship SR5 may be directedly determined without the tracker 75 based on virtual features of the virtual handheld surgical tool 21.

The navigation processor 207 also can determine a spatial relationship between the virtual localizer unit 54 and the virtual patient anatomy PA. In the examples of FIGS. 4 and 5, the virtual patient anatomy PA is represented using a virtual knee joint KJ of the virtual patient 12. As shown in FIGS. 4 and 5, the navigation processor 207 determines the spatial relationship SR7 between the virtual localizer unit 54 and the virtual knee joint KJ of the virtual patient 12. The navigation processor 207 determines the spatial relationship SR7 by determining a spatial relationship SR8 between the virtual localizer unit 54 and the virtual anatomy tracker 65 coupled to the virtual knee joint KJ of the virtual patient 12 and combining the determined spatial relationship SR8 with a known spatial relationship KSR4 between the virtual anatomy tracker 65 and the virtual knee joint KJ of the virtual patient 12. The spatial relationships SR7 and SR8 may be directedly determined without the trackers 64, 65, 66 and based on virtual features of the patient anatomy.

By combining any of the spatial relationships described above, the navigation processor 207 can know the pose of the virtual object relative to the virtual patient 12 and display the relationship between the virtual object and the virtual patient 12 on the virtual display device 24, 26.

The virtual reality processor 101 may determine a spatial relationship using any suitable method. For example, the virtual reality processor 101 may define a virtual coordinate system VECS of the virtual environment 11, the virtual coordinate system VECS being shown in FIGS. 4 and 5. The virtual coordinate system VECS may be bound by a geometry, such as a regular prism, as shown in FIG. 4. However, any other suitable size and shape of the virtual coordinate system VECS may be utilized. The virtual reality processor 101 may then determine coordinates of any two objects within the virtual environment 11 according to the virtual coordinate system VECS and compare the coordinates to determine the spatial relationship between the virtual surgical objects VSO. The two virtual surgical objects VSO may be any two virtual surgical objects VSO within the virtual environment 11. For example, a virtual surgical object VSO may be a virtual patient anatomy PA, the virtual localizer unit 54, the virtual manipulator 14, the virtual handheld surgical tool 21, the virtual pointer VP, a virtual surgical tool 16a, 16b, 16c, 16d, a virtual implant 18a, 18b, a virtual object surgical tracker 64, 66, 68, 72, 75, the virtual circulating table 20, a virtual participant 15, the virtual representation 17 of the user 13, and the like.

For example, referring to FIG. 4, the virtual reality processor 101 may determine the coordinates of the virtual localizer unit 54 and the coordinates of the virtual pointer VP to determine the spatial relationship SR1 between the virtual localizer unit 54 and the virtual pointer VP. As shown in FIG. 4, the virtual reality processor 101 may determine that the virtual localizer unit 54 includes coordinates (x_{lclz}, y_{lclz}, z_{lclz}) and that the virtual pointer

VP includes coordinates (x_{P}, y_{P}, z_{P}). The virtual reality processor 101 may then compare the coordinates (x_{lclz}, y_{lclz}, z_{lclz}) of the virtual localizer unit 54 and the coordinates (x_{P}, y_{P}, z_{P}) of the pointer VP relative to the virtual coordinate system VECS of the virtual environment 11 to determine the spatial relationship SR1 between the virtual localizer unit 54 and the virtual pointer.

The virtual reality processor 101 may display a virtual representation of a virtual surgical object VSO on a virtual display device 24, 26. The virtual reality processor 101 may display a virtual representation of any virtual surgical object VSO of the virtual environment 11, such as a virtual patient anatomy PA of the virtual patient 12, the virtual localizer unit 54, the virtual manipulator 14, the virtual handheld surgical tool 21, the virtual pointer VP, a virtual surgical tool 16a, 16b, 16c, 16d, a virtual implant 18a, 18b, the virtual circulating table 20, a virtual participant 15, the virtual representation 17 of the user, and the like.

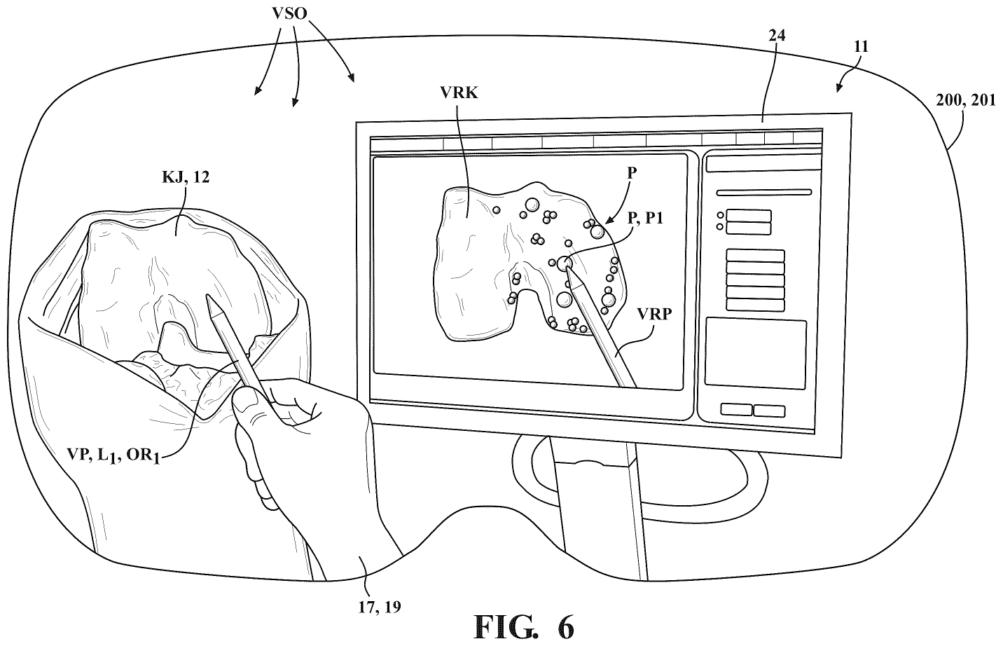

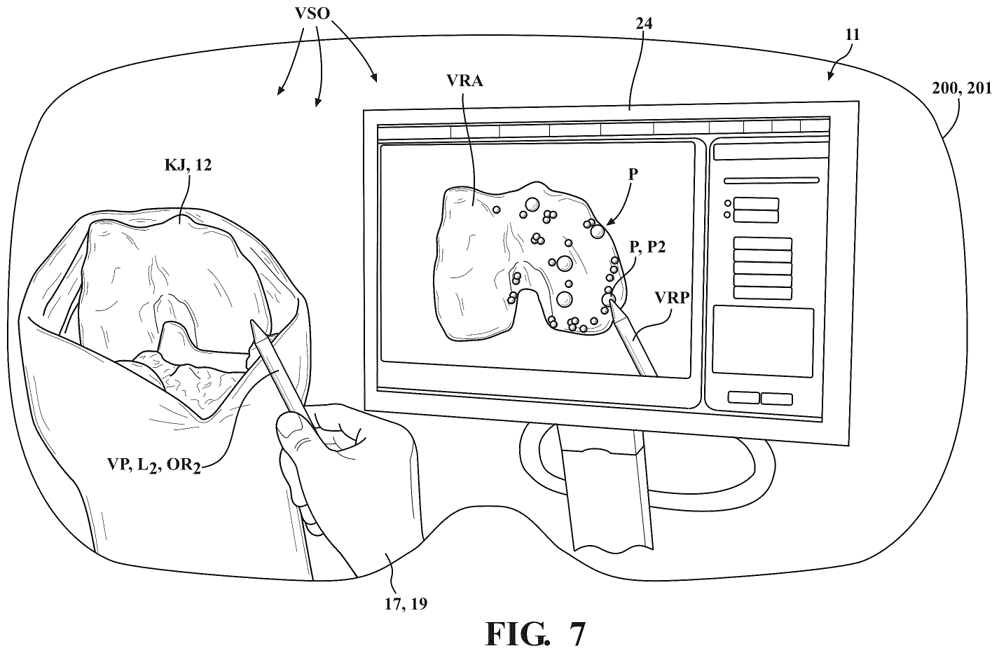

Referring to FIGS. 6 and 7, the virtual reality processor 101 may display a virtual representation of one or more virtual surgical objects VSO on a virtual display device 24, 26. In this way, the user 13 of the virtual reality surgical system 10 may view a virtual representation of one or more virtual surgical objects VSO when viewing the virtual environment 11 through the HMD 200. The virtual surgical object VSO may be any virtual surgical object VSO described herein. For example, in FIGS. 6 and 7, the virtual surgical objects VSO are the virtual pointer VP and a virtual patient anatomy PA, illustrated as the virtual knee joint KJ. As shown, a virtual representation VRP of the virtual pointer VP and a virtual representation VRA of the virtual patient anatomy PA are displayed on the virtual display 24. The virtual representation VRA of the virtual patient anatomy PA may be a virtual representation of any virtual patient anatomy PA. In the instance of FIGS. 6 and 7, the virtual surgical object VSO is a virtual knee joint KJ and the virtual representation VRA of the virtual patient anatomy PA is a virtual representation VRA of the virtual knee joint KJ.

Furthermore, the virtual reality processor 101 may be configured to display a virtual representation of a virtual surgical object VSO on a virtual display device 24, 26, wherein a pose (a location and/or orientation) of the virtual representation is based on the determined spatial relationship between the virtual localizer unit 54 and the virtual surgical object VSO in the virtual environment 11.

Referring to FIGS. 6 and 7, two virtual representations VRP of the virtual pointer VP are shown in the virtual display 24 at different points in time. In FIG. 6, the virtual pointer VP includes a first pose, which includes location L₁ and orientation OR₁ . In FIG. 7, the virtual pointer VP includes a second pose different from the first pose, the second pose including location L₂ and orientation OR₂. As the virtual pointer VP moves through the virtual environment 11, the pose of the virtual pointer VP changes, as does the spatial relationship SR1 between the virtual localizer unit 54 and the virtual pointer VP. Once the virtual reality processor 101 determines the pose of the virtual pointer VP based on the spatial relationship SR1, the virtual reality processor 101 may display the virtual representation VRP of the virtual pointer VP on the virtual display 24 in accordance with the determined pose. Accordingly, the user 13 is able to experience this user-guided aspect surgical navigation in the virtual reality world.

Additionally, the virtual reality processor 101 may be configured to display, on a virtual display device 24, 26 feedback related to a spatial relationship between virtual surgical objects VSO. For example, the virtual display device 24 may be configured to display a distance between the virtual localizer unit 54 and the virtual handheld surgical tool 21. As another example, the virtual display device 24 may be configured to display a distance between the virtual localizer unit 54 and the virtual tracker 75 coupled to the virtual handheld surgical tool 21.

### B. Determining Virtual Trackability

In some instances, the virtual reality processor 101 may determine a virtual trackability of a virtual surgical object VSO to the virtual localizer unit 54. The virtual trackability is the virtual assessment of determining whether the virtual surgical object VSO, or tracker attached thereto, would be trackable by the virtual localizer unit 54 in the virtual environment 11. The virtual trackability may be based the actual trackability or a simulated assessment of the trackability of the virtual object. Assuming the virtual localizer unit 54 is an optical or camera-based system, the virtual trackability can be understood as a virtual visibility of the surgical object or tracker respectively coupled thereto. The process of evaluating the virtual trackability can be performed during or before determining a spatial relationship between the virtual surgical object VSO and the virtual localizer unit 54 and displaying the virtual surgical object VSO on the virtual display unit 24, 26.

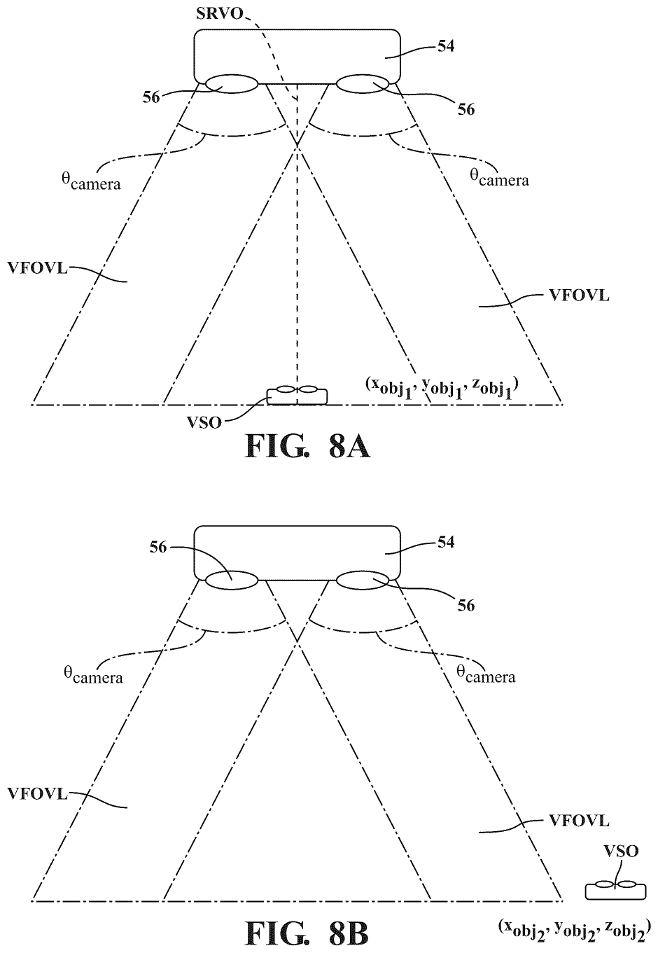

The trackability of a virtual surgical object VSO may be based on a virtual field of view VFOVL (see FIGS. 8A and 8B) of the virtual localizer unit 54. For example, the virtual reality processor 101 may generate the virtual field of view VFOVL such that virtual surgical objects VSO within the virtual field of view VFOVL are simulated as being detectable by the virtual localizer unit 54 and can displayed on the virtual display unit 24 as being detected. Similarly, the virtual reality processor 101 may generate the virtual field of view VFOVL such that objects not within the virtual field of VFOVL are simulated as being undetected by the virtual localizer unit 54. The virtual reality processor 101 can provide notifications or feedback to the user indicating the outcome of the virtual trackability assessment.

When non-optical virtual localizer units 54 are utilized, the techniques herein can be applied by using a virtual field of trackability, rather than a field of view. Any of the techniques described herein related to determining visibility of the surgical object can be applied to assessing the general trackability of the virtual object.

In one instance, the virtual reality processor 101 may determine a trackability of a virtual surgical object VSO based on coordinates of the virtual surgical object VSO relative to the virtual field of view VFOVL of the virtual localizer unit 54. In such an instance, the virtual reality processor 101 may determine that a virtual surgical object VSO is visible to the virtual localizer unit 54 once the virtual surgical object VSO enters the virtual field of view VFOVL. Similarly, the virtual reality processor 101 may determine that a virtual surgical object VSO is not visible to the virtual localizer unit 54 once the virtual surgical object VSO exits the virtual field of view VFOVL. A virtual boundary may be associated with the virtual field of view VFOVL and a virtual shape may be associated with the virtual object. Trackability can be evaluated by assessing whether the virtual shape exceeds, or is within, the virtual boundary of the VFOVL.

The virtual reality processor 101 may determine a visibility of a virtual surgical object VSO by determining the coordinates of the virtual localizer unit 54 and the coordinates of the virtual surgical object VSO relative to the virtual field of view VFOVL and comparing the coordinates of the virtual localizer unit 54 and the coordinates of the virtual surgical object VSO relative to the virtual field of view VFOVL. For example, in FIGS. 8A and 8B, the virtual reality processor 101 may determine the coordinates of the virtual localizer unit 54 to be (x_{lclz}, y_{lclz}, z_{lclz}) and may determine the coordinates within the virtual field of view VFOVL based on a visibility angle θ _{camera} of the virtual camera units 56 of the virtual localizer unit 54. In the instance of FIG. 8A, the virtual reality processor 101 may determine the coordinates of the virtual surgical object VSO to be (x_{obj1}, y_{obj1}, z_{obj1}). In the instance of FIG. 8B, the virtual reality processor 101 may determine the coordinates of the virtual surgical object VSO to be (x_{obj2}, y_{obj2}, z_{obj2}). The virtual reality processor 101 may then compare the coordinates of the virtual surgical object VSO with the coordinates within the virtual field of view VFOVL to determine the visibility of the virtual surgical object VSO. In FIG. 8A, the virtual reality processor 101 may determine the virtual surgical object VSO to be within the virtual field of view VFOVL and that the virtual surgical object VSO is visible to the virtual localizer unit 54. In the instance of FIG. 8B, the virtual reality processor 101 may determine the virtual surgical object VSO to not be within the virtual field of view VFOVL and that the virtual surgical object VSO is not visible to the virtual localizer unit 54.

In another instance, the virtual reality processor 101 may determine a trackability of a virtual surgical object VSO to the virtual localizer unit 54 based on a field of view VFOVO of the virtual surgical object VSO and the virtual field of view VFOVL of the virtual localizer unit 54. In such instances, the virtual reality processor 101 may determine that the virtual surgical object VSO is visible to the virtual localizer unit 54 once the virtual surgical object VSO enters the virtual field of view VFOVL of the virtual localizer unit 54 and the virtual localizer unit enters the virtual field of view VFOVO of the virtual surgical object VSO. Similarly, the virtual reality processor 101 may determine that the virtual surgical object VSO is not visible to the virtual localizer unit 54 once the virtual surgical object VSO exits the virtual field of view VFOVL of the virtual localizer unit 54 and/or the virtual localizer unit exits the virtual field of view VFOVO of the virtual surgical object VSO.

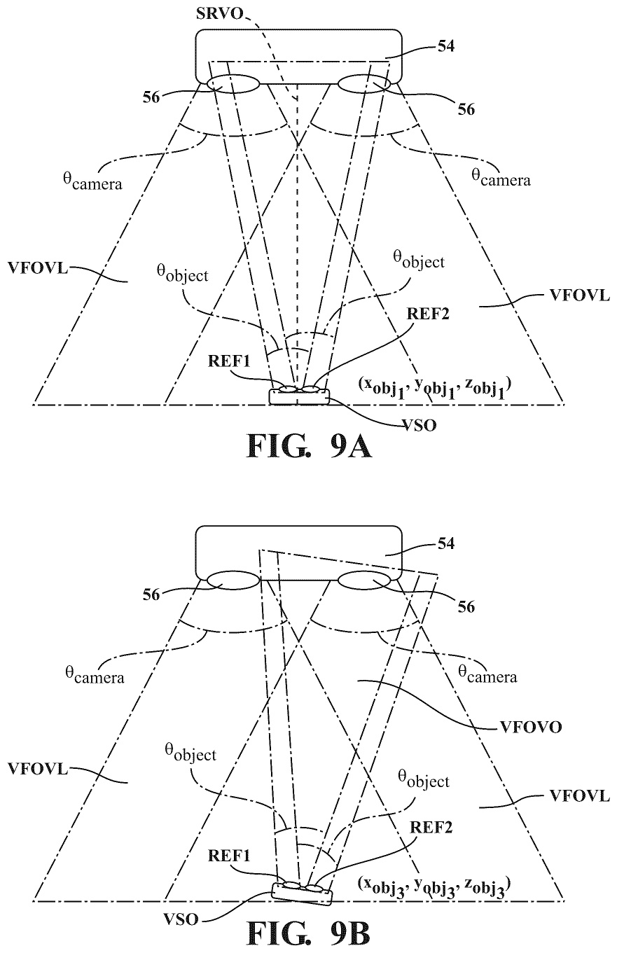

The virtual reality processor 101 may determine a trackability of a virtual surgical object VSO by determining the coordinates of the virtual localizer unit 54 relative to the virtual field of view VFOVO and the coordinates of the virtual surgical object VSO relative to the virtual field of view VFOVL. The virtual reality processor 101 may then compare the coordinates of the virtual localizer unit 54 and the coordinates of the virtual surgical object VSO relative to the virtual field of view VFOVL and relative to the virtual field of view VFOVO. For example, in FIGS. 9A and 9B, the virtual reality processor 101 may determine the coordinates of the virtual localizer unit 54 to be (x_{lclz}, y_{lclz}, z_{lclz}) and may determine the coordinates within the virtual field of view VFOVL based on a visibility angle θ_{camera} of the virtual camera units 56 of the virtual localizer unit 54. In the instance of FIG. 9A, the virtual reality processor 101 may determine the coordinates of the virtual surgical object VSO to be (x_{obj1}, y_{obj1}, z_{obj1}) and may determine the coordinates within the virtual field of view VFOVO based on a visibility angle θ_{object} of viewport points REF1, REF2 of the virtual surgical object VSO. In the instance of FIG. 9B, the virtual reality processor 101 may determine the coordinates of the virtual surgical object VSO to be (x_{obj3}, y_{obj3}, z_{obj3}) and may determine the coordinates within the virtual field of view VFOVO based on a visibility angle θ_{object} of the viewport points REF1, REF2 of the virtual surgical object VSO. The virtual reality processor 101 may then compare the coordinates of the virtual surgical object VSO with the coordinates within the virtual field of view VFOVL and may compare the coordinates of the virtual localizer unit 54 with the coordinates within the virtual field of view VFOVO to determine the visibility of the virtual surgical object VSO. In the instance of FIG. 9A, the virtual reality processor 101 may determine that the viewport points REF1, REF2 are within the virtual field of view VFOVL, that both virtual camera units 56 are within the virtual field of view VFOVO and, therefore, that the virtual surgical object VSO is visible to the virtual localizer unit 54. In the instance of FIG. 9B, the virtual reality processor 101 may determine that the viewport points REF1, REF2 are within the virtual field of view VFOVL, but that only one of the virtual camera units 56 is within the virtual field of view VFOVO and, therefore, that the virtual surgical object VSO is not visible to the virtual localizer unit 54.

The viewport points REF1, REF2 of a virtual surgical object VSO are points from which the virtual field of view VFOVO is generated by the virtual reality processor 101 for assessing a trackability of the virtual surgical object VSO. The viewport points REF1, REF2 may be any point on a surface of a virtual surgical object VSO. In some instances, the viewport points REF1, REF2 may be determined arbitrarily. In other instances, the viewport points REF1, REF2 may be customized based on the shape of the object. For example, the viewport points REF1, REF2 may be customized such that the viewport points REF1, REF2 are on a surface of the virtual surgical object VSO that is closest to the virtual localizer unit 54. In a more specific example, in instances where the virtual surgical object VSO is a virtual patient anatomy PA, viewport points REF1, REF2 may be customized such that the viewport points REF1, REF2 are on a surface of the virtual patient anatomy PA that the user 13 may interact with during use of the virtual reality surgical system 10.

It is contemplated that, in other instances, the virtual localizer unit 54 may include a greater or fewer number of virtual camera units 56. Similarly, it is contemplated that the virtual surgical object VSO may include a greater or fewer number of viewports REF1, REF2. As such, the virtual localizer unit 54 may include a virtual field of view VFOVL that varies from the virtual field of view VFOVL shown in FIGS. 8A-9B and the virtual surgical object VSO may include a virtual field of view VFOVO that varies from the virtual field of view VFOVO shown in FIGS. 9A and 9B.

Additionally, in the instances of FIG. 9A and 9B, the virtual reality processor 101 determines that the virtual surgical object VSO is visible to the virtual localizer unit 54 based on all of the viewports REF1, REF2 of the virtual surgical object VSO being within the virtual field of view VFOVL and all of the virtual camera units 56 of the virtual localizer unit 54 being within the virtual field of view VFOVO. However, in other instances, the virtual reality processor 101 may determine that the virtual surgical object VSO is visible to the virtual localizer unit 54 based on less than all of the viewports REF1, REF2 of the virtual surgical object VSO being within the virtual field of view VFOVL and less than all of the virtual camera units 56 of the virtual localizer unit 54 being within the virtual field of view VFOVO.

In another example, trackability may depend on virtual obstructions between the virtual object and the virtual localizer unit 54. For instance, when an optical virtual localizer unit 54 is utilized, the virtual reality processor 101 may determine that an obstructing virtual object interferes with a virtual line-of-sight between the virtual localizer unit 54 and the virtual object using any of the techniques described above. Once the obstructing virtual object no longer interferes with the virtual line-of-sight, the virtual trackability is restored. In some instances, a virtual line-of-sight boundary may be established between the virtual localizer unit 54 and the virtual object. A virtual shape may be associated with the virtual obstructing object. The virtual reality processor 101 may determine that the obstruction is present once the virtual shape of the obstructing object intersects the virtual line-of-sight boundary, and vice-versa.

In instances where the virtual reality processor 101 determines that the virtual surgical object VSO is trackable by the virtual localizer unit 54 and the virtual reality processor 101 can determine a spatial relationship between the virtual surgical object VSO and the virtual localizer unit 54, the virtual reality processor 101 is configured to provide feedback about this trackability by enabling functions related to virtual surgical navigation, such as displaying the virtual representation of the virtual surgical object VSO on the virtual display device 24, 26 at the respective tracked pose of the virtual surgical object VSO.

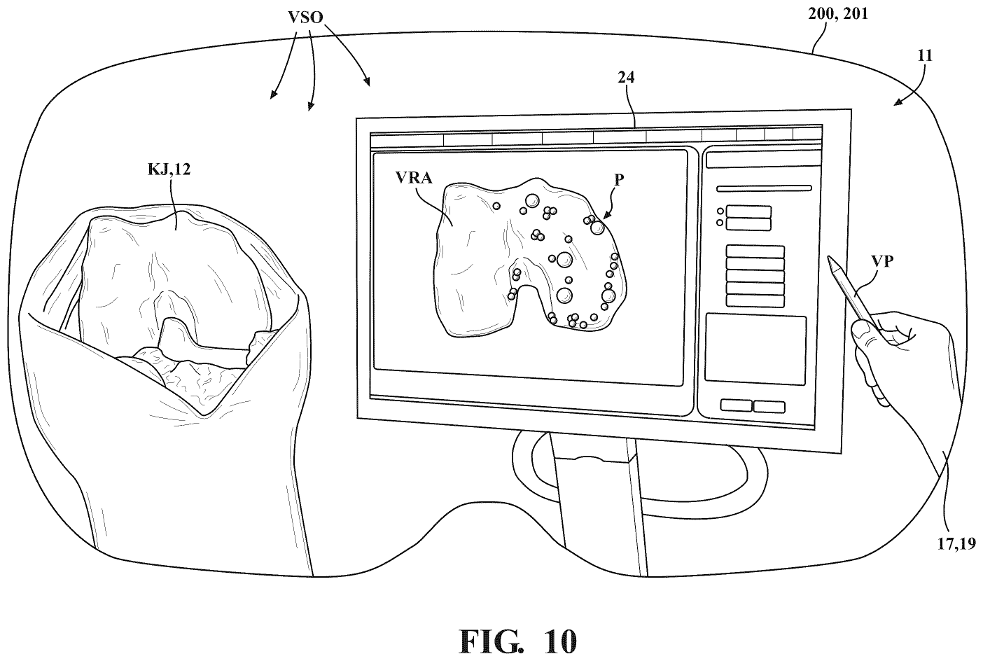

In instances where the virtual reality processor 101 determines that the virtual surgical object VSO is not visible to the virtual localizer unit 54 and the virtual reality processor 101 does not determines a spatial relationship between the virtual surgical object VSO and the virtual localizer unit 54, the virtual reality processor 101 is configured to provide feedback about this lack of trackability by disabling functions related to virtual surgical navigation, such as no longer displaying the virtual representation of the virtual surgical object VSO on the virtual display device 24, 26. For example, in the instance of FIG. 10, the virtual reality processor 101 determines that the virtual pointer VP is not visible to the virtual localizer unit 54 as the virtual pointer VP has exited the field of view VFOVL of the virtual localizer unit 54. In response, the virtual reality processor 101 prevents the virtual representation VRP of the virtual pointer VP from being displayed on the virtual display 24.

Additionally, the virtual reality processor 101 may be configured to display, on a virtual display device 24, 26 feedback related to a visibility of a virtual surgical object VSO to the virtual localizer 54. For example, the virtual display device 24 may be configured to indicate to the user 13 when a virtual surgical object VSO is no longer visible to the virtual localizer unit 54. As another example, the virtual display device 24 may be configured to indicate to the user 13 when a virtual surgical object VSO has become visible to the virtual localizer unit 54.

The trackability of an object, or lack thereof, is an aspect of surgical navigation that is advantageously simulated by the techniques described herein. As such, the user 13 can experience, in the virtual-world, accurate and complete operation of the surgical navigation system.

### V. Example Uses of the Virtual Reality Surgical System

As will be described herein, the virtual reality surgical system 10 may be used to enable a user 13 to perform various surgical functions in the virtual world. For example, the virtual reality surgical system 10 may be used to enable the user 13 to register a virtual patient anatomy PA of the virtual patient 12 to the virtual navigation system 40 within the virtual environment 11. The virtual reality surgical system 10 may be used to enable the user 13 to perform cutting of a virtual patient anatomy PA of the virtual patient 12 within the virtual environment 11 using the virtual handheld surgical tool 21 and/or the virtual manipulator 14 (depending on whether one or both are used to perform the cutting).

For both of these purposes, the navigation processor 207 first determines a spatial relationship between the virtual localizer unit 54 and the corresponding virtual surgical object VSO in the virtual environment 11, as described above. In instances where the user 13 is registering a virtual patient anatomy PA, the virtual surgical object VSO may be the virtual pointer VP. In instances where the user 13 is performing cutting of a virtual patient anatomy

PA of the virtual patient 12, the virtual surgical object VSO may be the virtual manipulator 14 and/or the virtual handheld surgical tool 21.

### A. Virtual Patient Anatomy Registration

FIGS. 6 and 7 illustrate instances where the virtual reality surgical system 10 may be used to train a user 13 to register an anatomy of a patient to a surgical navigation system. The virtual reality surgical system 10 allows the user 13 to register a virtual patient anatomy PA of the virtual patient 12, represented as a virtual knee joint KJ in FIGS. 6 and 7, to the virtual navigation system 40 within the virtual environment 11. In other instances, the virtual patient anatomy PA may be any virtual patient anatomy PA of the virtual patient 12, such as the virtual femur F and/or the virtual tibia T.

As previously stated, the virtual reality processor 101 may display a virtual representation VRA of a virtual patient anatomy PA on a virtual display device 24, 26. Additionally, the virtual reality processor 101 may display points P to be collected on the surface of the virtual representation VRA of the virtual patient anatomy PA on a virtual display device 24, 26 for registering the virtual patient anatomy PA. In the instance of FIGS. 6 and 7, the display processor 210 displays on the virtual display 24 a virtual representation VRA of the virtual knee joint KJ and points P to be collected on the surface of the virtual representation VRA of the virtual knee joint KJ. In this way, the user 13 of the virtual reality surgical system 10 may view a virtual representation VRA of the virtual patient anatomy PA as well as points P to be collected on the surface of the virtual representation VRA of the virtual patient anatomy PA while registering the virtual patient anatomy PA.

The user 13 may register the virtual patient anatomy PA of the virtual patient 12 to the virtual navigation system 40 by controlling a position of the virtual probe, such as the virtual pointer VP, to collect the points P displayed on the virtual display 24 within the virtual environment 11. In order for the user 13 to control the position of the virtual pointer VP, the HMD 200 first receives an input from the user 13. The input from the user 13 may be a desired motion of the virtual pointer VP and may be received via a previously described user input device 203, via the tracking the hand of the user 13 (as described above and as shown in FIG. 3), and/or any other suitable means of receiving a desired movement from the user 13. For example, in the instance of FIG. 6, the display processor 210 receives an input from the user 13 corresponding to detecting a desired motion of the virtual pointer VP to a point P1 on the virtual knee joint KJ. In the instance of FIG. 7, the display processor 210 receives an input from the user 13 corresponding to detecting a desired motion of the virtual pointer VP to a point P2 on the virtual knee joint KJ.

Once the display processor 210 receives the input from the user, the display processor 210 may control a position of the virtual pointer VP based on the input from the user 13 and the virtual pointer VP may collect points on the surface of the virtual patient anatomy PA for registration. For example, in the instance of FIG. 6, the display processor 210 moves the virtual pointer VP in the virtual hand 19 to a point P1 on the virtual knee joint KJ to collect the point P1 for registration. In the instance of FIG. 7, the display processor 210 moves the virtual pointer VP in the virtual hand 19 to a point P2 on the virtual knee joint KJ to collect the point P2 for registration. The display processor 210 is configured to display on a virtual display device 24, 26 a virtual representation of the virtual patient anatomy PA and during collection of points P on the surface of the virtual patient anatomy PA. As shown, a virtual representation VRP of the virtual pointer VP is shown in FIGS. 6 and 7 as the virtual pointer VP is moved to points P1 and P2. In this way, the virtual reality processor 101 may register the virtual patient anatomy PA within the virtual environment 11 in response to the virtual pointer VP collecting points on a surface of the virtual patient anatomy PA based on the input from the user 13. Any type of virtual imageless surface registration may be implemented by the virtual reality surgical system. Furthermore, virtual registration as described herein may be customized based on a surgical plan specific to the virtual patient.

Furthermore, a haptic device may be configured to provide haptic feedback to the user 13 in response to the virtual pointer P collecting points P on the surface of the virtual patient anatomy PA. Similarly, an auditory device may be configured to provide audio feedback to the user 13 in response to the virtual pointer P collecting points P on the surface of the virtual patient anatomy PA. Additionally, the haptic and auditory device may be configured to provide haptic and audio feedback to the user 13 in response to the virtual pointer P completing registration of the virtual patient anatomy PA, in response to the virtual pointer P registering the virtual patient anatomy PA, and/or in response to the user 13 initiating registration of the virtual patient anatomy PA. The haptic and auditory device may be a haptic and auditory device of the HMD 200 and/or a haptic and auditory device of the user input device 203.

### B. Virtual Patient Anatomy Preparation

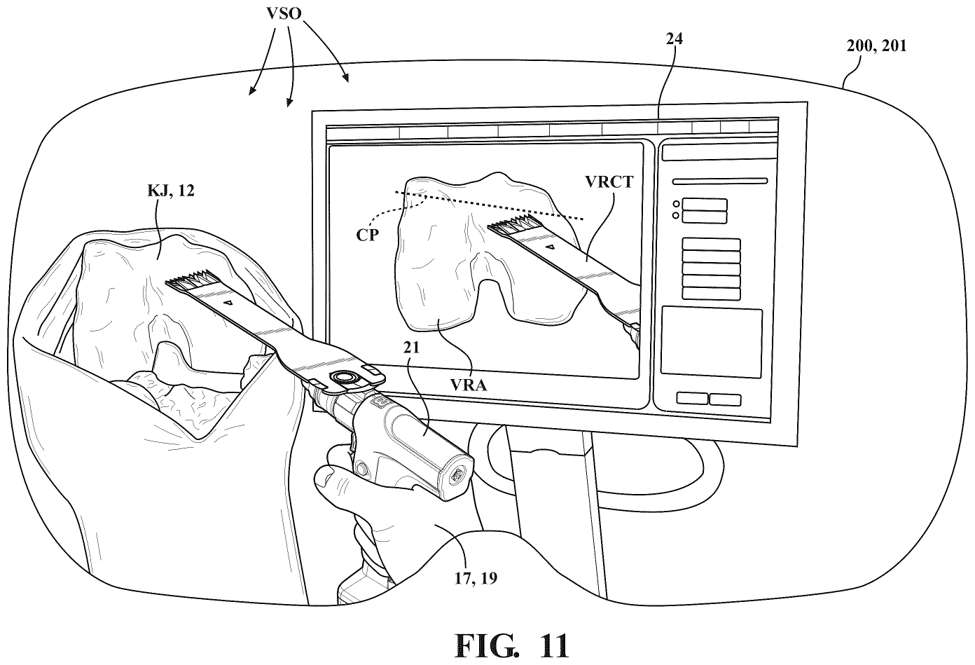

FIG. 11 illustrates an instance where the virtual reality surgical system 10 may be used to train a user 13 perform cutting of a virtual patient anatomy PA of the virtual patient 12, represented as a virtual knee joint KJ in FIG. 11, within the virtual environment 11. In other instances, the virtual patient anatomy PA may be any virtual patient anatomy PA of the virtual patient 12, including but not limited to the example anatomies described above.

The user 13 may perform cutting of the virtual patient anatomy PA of the virtual patient 12 by controlling a position and an operation of a virtual cutting tool, such as the virtual handheld surgical tool 21 within the virtual environment 11. Additionally, the virtual reality processor 101 may enable the virtual cutting tool to perform a virtual cutting of the virtual patient anatomy PA based on user control of the virtual cutting tool. In other instances, the virtual cutting tool may be any virtual surgical object VSO within the virtual environment 11 suitable for performing cutting of patient anatomy, such as the virtual manipulator 14.

In order for the user 13 to control the position of the virtual handheld surgical tool 21, the HMD 200 first receives an input from the user 13. The input from the user 13 may be a desired movement and operation of the virtual handheld surgical tool 21 and may be received via a previously described user input device 203, via the tracking the hand of the user 13 (as described above and as shown in FIG. 3), and/or any other suitable means of receiving a desired movement and operation from the user 13. For example, the display processor 210 may receive an input from the user 13 corresponding to detecting a desired motion of the virtual handheld surgical tool 21. As shown in FIG. 11, the desired motion of the virtual handheld surgical tool 21 may be to move the virtual handheld surgical tool 21 toward the virtual knee joint KJ. Additionally, the display processor 210 may receive an input from the user 13 to enable or deactivate the virtual handheld surgical tool 21. In instances where the display processor 210 receives an input from the user 13 to enable the virtual handheld surgical tool 21, the virtual reality processor 101 may enable the virtual handheld surgical tool 21, allowing the user 13 to perform a virtual cutting of the virtual knee joint KJ.

The display processor 210 is configured to display on a virtual display device 24, 26 a virtual representation of the virtual cutting tool relative to the virtual representation of the virtual patient anatomy PA during virtual cutting of the virtual patient anatomy PA. As shown in FIG. 11, the virtual display 24 displays a virtual representation VRCT of the virtual handheld surgical tool 21 relative to the virtual representation VRA of the virtual knee K during the virtual cutting of the virtual knee K by the virtual handheld surgical tool 21.

Additionally, the display processor 210 may be configured to display feedback related to the virtual cutting of the virtual patient anatomy PA on a virtual display device 24, 26. For example, referring to FIG. 11, the virtual display 24 displays a cutting path CP for guiding cutting of the virtual knee K with the virtual handheld surgical tool 21. The virtual display device 24, 26 may also display instructions to the user 13, such as a desired adjustment of the virtual handheld surgical tool 21 or virtual manipulator 14. The virtual display device 24, 26 may also display a cut plan of the virtual manipulator 14 to the user 13. The virtual display device 24, 26 may also display an indication that a cutting of the virtual patient anatomy PA has been initiated and/or completed.

The virtual reality processor 101 may be configured to define a virtual boundary relative to the virtual patient anatomy PA. The virtual boundary may delineate a region of the virtual patient anatomy PA to be cut by the virtual cutting tool from another region of the virtual patient anatomy PA to be avoided by the virtual cutting tool. The virtual reality processor 101 may also be configured to detect that the virtual cutting tool has met or exceed the virtual boundary. The virtual reality processor 101 may define the virtual boundary in instances where the user 13 performs cutting of the virtual patient anatomy PA using the virtual handheld surgical tool 21 and in instances where the user 13 performs cutting of the virtual patient anatomy PA using the virtual manipulator 14.

In instances where the virtual manipulator 14 performs cutting of the virtual patient anatomy PA, motion of the virtual manipulator 14 may be constrained by the virtual boundary. For example, a cut path for the virtual manipulator 14 may be defined based on the virtual boundary and the virtual reality processor 101 may control motion of the virtual manipulator 14 based on the cut path.

In instances where the virtual handheld surgical tool 21 performs cutting of the virtual patient anatomy PA, a haptic device may be configured to provide haptic feedback to the user 13 in response to the virtual reality processor 101 detecting that the virtual handheld cutting tool has met or exceed the virtual boundary. Similarly, an auditory device may be configured to provide audio feedback to the user 13 in response to the virtual reality processor 101 detecting that the virtual handheld surgical tool 21 has met or exceed the virtual boundary.

Additionally, the haptic and auditory device may be configured to provide haptic and audio feedback to the user 13 in response to the virtual handheld surgical tool 21 completing cutting of the virtual patient anatomy PA, in response to the virtual handheld surgical tool 21 performing cutting of the virtual patient anatomy PA, and/or in response to the virtual handheld surgical tool 21 initiating cutting of the virtual patient anatomy PA. The haptic and auditory device may be a haptic and auditory device of the HMD 200 and/or a haptic and auditory device of the user input device 203.

The display processor 210 may be configured to display the virtual boundary. For example, the cutting path CP shown on the virtual display 24 may be generated based on defined virtual boundaries. In other instances, the virtual display 24 may display a cut plan for the virtual manipulator 14, which may be generated based on the virtual boundary. In other instances, the virtual display 24 may display the region of the virtual patient anatomy PA to be cut and the region of the virtual patient anatomy PA to be avoided.

The display processor 210 may be configured to modify the virtual representation VRA of the virtual patient anatomy PA during cutting of the virtual patient anatomy PA. For example, after a portion of the virtual patient anatomy VRA has been removed during cutting, the display processor 210 may remove a corresponding portion from the virtual representation VRA of the virtual patient anatomy PA. The removed portion of the virtual patient anatomy VRA may be determined based on comparing coordinates of virtual patient anatomy PA and coordinates of the virtual handheld cutting tool 21 in the virtual coordinate system VECS of the virtual environment 11. The virtual reality processor 101 may then remove the removed portion from the virtual patient anatomy PA and the display processor 210 may modify the virtual representation VRA of the virtual patient anatomy PA to reflect that the removed portion has been removed from the virtual patient anatomy PA.

### C. Virtual Surgical Operating Room Preparation

The virtual reality surgical system 10 may include various configurations for training or enabling a user 13 to virtually prepare the virtual operating room for surgery.

In one configuration, the display 201 of the HMD 200 may be configured to display instructions for assembling a virtual surgical object VSO. For example, the HMD 200 may be configured to display instructions for assembly the virtual manipulator 14, the virtual handheld surgical tool 21, the virtual localizer unit 54, and/or any other virtual surgical object VSO described herein.

In another configuration, the display processor 210 may be configured to position a virtual surgical object VSO, such as the virtual handheld surgical tool 21, based on a position of a hand of the user 13. In one instance, a camera having a field of view may be configured to capture image data of a hand of the user and the display processor 210 may be configured to position the virtual surgical object VSO in the virtual environment 11 based on the image data of the hand of the user 13. The camera may be integrated into the HMD 200 or be separate from the HMD 200. For example, the display processor 210 may be configured to position the virtual surgical object VSO in the virtual representation hand 19 of the user 13 based on a position of the hand of the user 13. In a specific instance, the camera may capture image data of the hand of the user 13 and track an origin OH of the hand of the user 13 in a coordinate system HCS of the HMD 200 (shown in FIG. 3). The display processor 210 may then assign the origin OH of the hand to an origin OVSO of the virtual surgical object VSO in the virtual coordinate system VECS of the virtual environment 11 such that a position of the virtual surgical object VSO in the virtual coordinate system VECS corresponds to the captured position of the hand of the user 13 in the HMD coordinate system HCS. Additionally, the display processor 101 may assign the origin OH of the hand to an origin of the virtual hand 19 of the user 13 in the virtual coordinate system VECS such that the virtual surgical object VSO is positioned in the virtual hand 19 of the user 13 in the virtual environment 11.

In another configuration, a sensor may be configured detect motion of the hand of the user 13 and the display processor 210 may be configured to position a virtual surgical object VSO, such as the virtual handheld surgical tool 21, in the virtual environment 11 based on the detected motion of the hand of the user 13. The sensor may be integrated into the HMD 200 and/or the user input device 203, or the sensor may be separate from the HMD 200 and the user input device 203. For example, the display processor 210 may be configured to position the virtual surgical object VSO in the virtual representation hand 19 of the user 13 based on the detected motion of the hand. In a specific instance, the sensor may track an origin OH of the hand of the user 13 in a coordinate system HCS of the HMD 200 (shown in FIG. 3) based on the detected motion. The display processor 101 may then assign the origin OH of the hand to an origin OVSO of the virtual surgical object VSO in the virtual coordinate system VECS of the virtual environment 11 such that a position of the virtual surgical object VSO in the virtual coordinate system VECS corresponds to the position of the hand of the user 13 in the HMD coordinate system HCS. Additionally, the display processor 210 may assign the origin OH of the hand to an origin of the virtual hand 19 of the user 13 in the virtual coordinate system VECS such that the virtual surgical object VSO is positioned in the virtual hand 19 of the user 13 in the virtual environment 11.

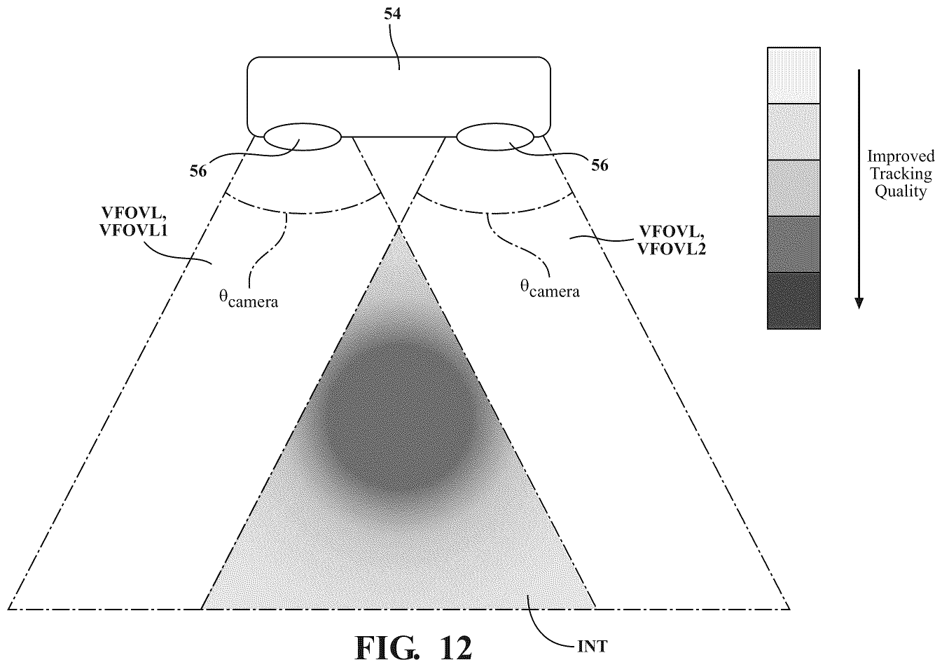

In another configuration, the user 13 may prepare the virtual environment 11 based on a tracking quality of the virtual localizer unit 54, which may be determined by the virtual reality processor 101. Referring to FIG. 12, the virtual localizer unit 54 includes a virtual field of view VFOVL, which may include a first virtual field of view VFOVL1 and a second virtual field of view VFOVL2, each being centered about a virtual camera unit 56 of the virtual localizer unit 54. Also shown, tracking quality varies within an intersection INT of the first virtual field of view VFOVL1 and a second virtual field of view VFOVL2, mimicking potential tracking capabilities of an actual localizer unit. In some instances, the user 13 of the virtual reality processor 101 may position virtual surgical objects VSO, such as the virtual manipulator 14 and/or the virtual handheld surgical tool 21, within the virtual environment 11 such that the virtual surgical objects VSO are located within the intersection INT of the first virtual field of view VFOVL1 and a second virtual field of view VFOVL2. Additionally, the user 13 may position the virtual localizer unit 54 such that virtual surgical objects VSO within the virtual environment 11 are positioned within the intersection INT. The virtual reality processor 101 may provide feedback to the user 13 via the virtual display 24, the HMD 200, and/or the user input device 203 indicating whether virtual surgical objects VSO within the virtual environment 11 are positioned within the intersection INT.

Each of the controllers have one or more processors, microprocessors, microcontrollers, field programmable gate arrays, systems on a chip, discrete circuitry, and/or other suitable hardware, software, or firmware that is capable of carrying out the functions described herein. The controllers may communicate with a network via wired connections and/or one or more communication devices, which may be wireless transceivers that communicate via one or more known wireless communication protocols such as WiFi, Bluetooth, Zigbee, and the like. The controllers may be connected in any suitable manner, including in a distributed network architecture, to a bus (e.g., a controller area network), and/or one or more of the controllers may be on separate networks that communicate with each other. In some cases, the function recited as being performed by the controllers may be performed by other controllers or by a single controller. For example, the workflow controller WC may comprise any one or more of the navigation controller, the machine vision controller, the projector controller, and the manipulator controller.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A virtual reality surgical system, comprising:
a head-mounted device comprising a display positionable in front of eyes of a user; and
at least one processor configured to:
provide, on the display of the head-mounted device, a virtual environment and a virtual surgical object, a virtual display device, and a virtual navigation system located within the virtual environment, the virtual navigation system including a virtual localizer unit;
determine a spatial relationship between the virtual localizer unit and the virtual surgical object in the virtual environment; and
display a virtual representation of the virtual surgical object on the virtual display device, wherein a pose of the virtual representation is based on the determined spatial relationship between the virtual localizer unit and the virtual surgical object in the virtual environment.

2. The virtual reality surgical system of claim 1, comprising the at least one processor configured to:
provide the virtual navigation system to further include a virtual surgical object tracker coupled to the virtual surgical object within the virtual environment; and
determine the spatial relationship between the virtual localizer unit and the virtual surgical object in the virtual environment by being configured to combine a first spatial relationship determined between the virtual localizer unit and the virtual surgical object tracker with a known spatial relationship between the virtual surgical object tracker and the virtual surgical object.

3. The virtual reality surgical system of claim 1, comprising the at least one processor configured to:
further provide, on the display of the head-mounted device, a virtual patient anatomy located within the virtual environment;
determine a spatial relationship between the virtual localizer unit and the virtual patient anatomy in the virtual environment; and
display a virtual representation of the virtual patient anatomy on the virtual display device.

4. The virtual reality surgical system of claim 3, comprising the at least one processor configured to receive image data of an actual patient anatomy and to provide the virtual patient anatomy based on the image data of the actual patient anatomy.

5. The virtual reality surgical system of claim 3, comprising the at least one processor configured to:
provide the virtual navigation system to further include a virtual anatomy tracker coupled to the virtual patient anatomy within the virtual environment; and
determine the spatial relationship between the virtual localizer unit and the virtual patient anatomy in the virtual environment by being configured to combine a first spatial relationship determined between the virtual localizer unit and the virtual anatomy tracker with a known spatial relationship between the virtual anatomy tracker and the virtual patient anatomy.

6. The virtual reality surgical system of claim 1, wherein the at least one processor is configured to:
define a coordinate system of the virtual environment;
determine coordinates of the virtual localizer unit and coordinates of the virtual surgical object relative to the coordinate system of the virtual environment; and
determine the spatial relationship between the virtual localizer unit and the virtual surgical object by being configured to compare the coordinates of the virtual localizer unit and coordinates of the virtual surgical object relative to the coordinate system of the virtual environment.

7. The virtual reality surgical system of claim 1, wherein the at least one processor is configured to:
provide the virtual localizer unit with a virtual field of view;
determine coordinates of the virtual localizer unit and coordinates of the virtual surgical object relative to the virtual field of view; and
determine the spatial relationship between the virtual localizer unit and the virtual surgical object by being configured to compare the coordinates of the virtual localizer unit and coordinates of the virtual surgical object relative to the virtual field of view.

8. The virtual reality surgical system of claim 7, wherein the at least one processor is configured to:
display the virtual representation of the virtual surgical object on the virtual display device in response to the virtual surgical object entering the virtual field of view of the virtual localizer unit; and
prevent the display of the virtual representation of the virtual surgical object on the virtual display device in response to the virtual surgical object exiting the virtual field of view of the virtual localizer unit.

9. The virtual reality surgical system of claim 3, wherein the virtual surgical object is further defined as a virtual probe, and wherein the at least one processor is configured to:
receive an input from the user to control a position of the virtual probe within the virtual environment; and
register the virtual patient anatomy in response to the virtual probe collecting points on a surface of the virtual patient anatomy based on the input from the user.

10. The virtual reality surgical system of claim 9, wherein the at least one processor is configured to:
display, on the virtual display device, the virtual representation of the virtual patient anatomy, and points to be collected on the surface of the virtual representation of the virtual patient anatomy;
display, on the virtual display device, the virtual representation of the virtual surgical object relative to the virtual representation of the virtual patient anatomy during collection of points on the surface; and
display, on the virtual display device, a notification or alert indicative of completion of a proper registration of the virtual patient anatomy.

11. The virtual reality surgical system of claim 9, further comprising a haptic device configured to provide haptic feedback to the user in response to the virtual probe collecting points on the surface of the virtual patient anatomy.

12. The virtual reality surgical system of claim 3, wherein the virtual surgical object is further defined as a virtual cutting tool, and wherein the at least one processor is configured to:
receive an input from the user to control a position and an operation of the virtual cutting tool within the virtual environment; and
enable the virtual cutting tool to perform a virtual cutting of the virtual patient anatomy based on user control of the virtual cutting tool.

13. The virtual reality surgical system of claim 12, wherein the at least one processor is configured to:
display, on the virtual display device, the virtual representation of the virtual cutting tool relative to the virtual representation of the virtual patient anatomy during the virtual cutting of the virtual patient anatomy; and
display, on the virtual display device, feedback related to the virtual cutting of the virtual patient anatomy.

14. The virtual reality surgical system of claim 12, wherein the virtual cutting tool is further defined as a virtual hand-held cutting tool or a virtual robotic manipulator.

15. The virtual reality surgical system of claim 12, further comprising a haptic device configured to provide haptic feedback to the user, and wherein the at least one processor is configured to:
define a virtual boundary relative to the virtual patient anatomy, the virtual boundary delineating a region of the virtual patient anatomy to be cut by the virtual cutting tool from another region of the virtual patient anatomy to be avoided by the virtual cutting tool; and
detect that the virtual cutting tool has met or exceeded the virtual boundary; and
in response, cause the haptic device to provide haptic feedback to the user.

16. The virtual reality surgical system of claim 1, wherein the virtual environment is a virtual surgical operating room.

17. The virtual reality surgical system of claim 1, further comprising a camera having a field of view to capture image data of a hand of the user, and wherein the at least one processor is configured to position the virtual surgical object in the virtual environment based on the image data of the hand of the user.

18. The virtual reality surgical system of claim 1, further comprising a user input configured to detect a motion of a hand of the user, and wherein the at least one processor is configured to position the virtual surgical object in the virtual environment based on the detected motion of the hand of the user.

19. The virtual reality surgical system of claim 1, comprising the at least one processor configured to:
provide the virtual navigation system to further include a virtual tracker within the virtual environment;
determine the spatial relationship between the virtual localizer unit and the virtual tracker in the virtual environment; and
display, on the virtual display device, feedback related to the spatial relationship between the virtual localizer unit and the virtual tracker in the virtual environment.

20. A method of operating a virtual reality surgical system, the virtual reality surgical system comprising a head-mounted device including a display positionable in front of eyes of a user, and at least one processor, the method comprising the at least one processor performing steps of:
providing, on the display of the head-mounted device, a virtual environment and a virtual surgical object, a virtual display device, and a virtual navigation system located within the virtual environment, the virtual navigation system including a virtual localizer unit;
determining a spatial relationship between the virtual localizer unit and the virtual surgical object in the virtual environment; and
displaying a virtual representation of the virtual surgical object on the virtual display device, wherein a pose of the virtual representation is based on the determined spatial relationship between the virtual localizer unit and the virtual surgical object in the virtual environment.

21. The method of claim 20, wherein the virtual surgical object is further defined as a virtual tool, and comprising the at least one processor:
further providing, on the display of the head-mounted device, a virtual patient anatomy located within the virtual environment;
displaying a virtual representation of the virtual patient anatomy on the virtual display device;
receiving an input from the user to control a position of the virtual tool within the virtual environment; and
registering the virtual patient anatomy in response to the virtual tool collecting points on a surface of the virtual patient anatomy based on the input from the user.

22. The method of claim 20, wherein the virtual surgical object is further defined as a virtual tool, and comprising the at least one processor:
further providing, on the display of the head-mounted device, a virtual patient anatomy located within the virtual environment;
displaying a virtual representation of the virtual patient anatomy on the virtual display device;
receiving an input from the user to control a position and an operation of the virtual tool within the virtual environment; and
enabling the virtual tool to virtually manipulate the virtual patient anatomy based on user control of the virtual tool.

23. The method of claim 20, comprising the at least one processor:
providing the virtual localizer unit with a virtual field of view;
displaying the virtual representation of the virtual surgical object on the virtual display device in response to the virtual surgical object entering the virtual field of view of the virtual localizer unit; and
preventing the display of the virtual representation of the virtual surgical object on the virtual display device in response to the virtual surgical object exiting the virtual field of view of the virtual localizer unit.

24. A non-transitory computer readable medium being configured to implement a virtual reality surgical system, the virtual reality surgical system comprising a head-mounted device comprising a display positionable in front of eyes of a user, and at least one processor, wherein the non-transitory computer readable medium comprises instructions, which when executed by the at least one processor, are configured to:
provide, on the display of the head-mounted device, a virtual environment and a virtual surgical object, a virtual display device, and a virtual navigation system located within the virtual environment, the virtual navigation system including a virtual localizer unit;
determine a spatial relationship between the virtual localizer unit and the virtual surgical object in the virtual environment; and
display a virtual representation of the virtual surgical object on the virtual display device, wherein a pose of the virtual representation is based on the determined spatial relationship between the virtual localizer unit and the virtual surgical object in the virtual environment.

25. The non-transitory computer readable medium of claim 24, wherein the virtual surgical object is further defined as a virtual tool, and wherein the instructions, when executed by the at least one processor, are configured to:
further provide, on the display of the head-mounted device, a virtual patient anatomy located within the virtual environment;
display a virtual representation of the virtual patient anatomy on the virtual display device;
receive an input from the user to control a position of the virtual tool within the virtual environment; and
register the virtual patient anatomy in response to the virtual tool collecting points on a surface of the virtual patient anatomy based on the input from the user.

26. The non-transitory computer readable medium of claim 24, wherein the virtual surgical object is further defined as a virtual tool, and wherein the instructions, when executed by the at least one processor, are configured to:
further provide, on the display of the head-mounted device, a virtual patient anatomy located within the virtual environment;
display a virtual representation of the virtual patient anatomy on the virtual display device;
receive an input from the user to control a position and an operation of the virtual tool within the virtual environment; and
enable the virtual tool to virtually manipulate the virtual patient anatomy based on user control of the virtual tool.

27. The non-transitory computer readable medium of claim 24, wherein the instructions, when executed by the at least one processor, are configured to:
provide the virtual localizer unit with a virtual field of view;
display the virtual representation of the virtual surgical object on the virtual display device in response to the virtual surgical object entering the virtual field of view of the virtual localizer unit; and
prevent the display of the virtual representation of the virtual surgical object on the virtual display device in response to the virtual surgical object exiting the virtual field of view of the virtual localizer unit.
